(19) United States
(12) Patent Application Publication
(10) Pub. No.: US 2004/0249577 A1
(43) Pub. Date: Dec. 9, 2004

(54) Method and apparatus for identifying components of a system with a response acteristic

(76) Inventors: Harri Kiiveri, Bull Creek Wa (AU); Mervyn Thomas, Chapel Hill (AU); Dale Wilson, Lilyfield (AU); Robert Dunne, NSW (AU)

Correspondence Address: LADAS & PARRY, 5670 WILSHIRE BOULEVARD, SUITE 2100, LOS ANGELES, CA, 90036-5679, US

(21) Appl. No.: 10/483,704

(22) PCT Filed: Jul. 11, 2002
(86) PCT No.: PCT/AU02/00934

(30) Foreign Application Priority Data
Jul. 11, 2001 (AU) PR 6316

(51) Int. Cl. G06F 19/00; G01N 33/48; G01N 33/50

(52) U.S. Cl. 702/20

(57) ABSTRACT

A method for identifying components of a system from data generated from the system, which exhibit a response pattern associated with a test condition applied to the system, comprising the steps of specifying design factors to specify a response pattern for the test condition and identifying a linear combination of components from the input data which correlate with the response pattern.

### TECHNICAL FIELD OF THE INVENTION

The invention relates to a method and apparatus for identifying components of a system from data generated from the system, which components are capable of exhibiting a response pattern associated with a test condition and, particularly, but no exclusively, the present invention relates to a method and apparatus for identifying components of a biological system from data generated from the system, which components are capable of exhibiting a response pattern associated with a test condition.

### BACKGROUND OF THE INVENTION

There are any number of “systems” in existence for which measurement of components of the system may provide a basis by which to analyse the system. Examples of systems include financial systems (such as stock markets, credit systems for individuals, groups, organisations, loan histories), geological systems, chemical systems, biological systems, and many more. Many of these systems comprise a substantial number of components which generate substantial amounts of data.

For example, recent advances in the biological sciences have resulted in the development of methods for large scale analysis of biological systems. An example of one such method is use of biotechnology arrays. These arrays are generally ordered high density grids of known biological samples (e.g. DNA, protein, carbohydrate) which may be screened or probed with test samples to obtain information about the relative quantities of individual components in the test sample. Use of biotechnology arrays thus provides potential for analysis of biological and/or chemical systems.

An example of one type of biotechnology array is DNA microarrays for the analysis of gene expression. A DNA microarray consists of DNA sequences deposited in an ordered array onto a solid support base e.g. a glass slide. As many as 30,000 or more gene sequences may be deposited onto a single microarray chip. The arrays are hybridised with labelled RNA extracted from cells or tissue of interest, or cDNA synthesised from the extracted RNA, to determine the relative amounts of the RNA expression for each gene in the cell or tissue. The technique therefore provides a method of determining the relative expression levels of many genes in a particular cell or tissue. The method also has the potential to allow for the identification of genes that are expressed in a particular way, or in other words, have a particular response pattern in different cell types, or in the same cell type under different treatment or test conditions.

The ability to identify such genes would be useful, for example, in establishing diagnostic tests to distinguish between different cell types, to determine optimum conditions for expression of desired genes, or in assessing efficacy of drugs for targeting expression of particular genes.

A significant problem with the analysis of data generated from systems such as biotechnology arrays, however, is that response patterns in the data are often difficult to identify due to one or more of the following:

(a) the difficulty in manipulating large amounts of data generated by these types of methods or experiments;

(b) the inherent variation in the data;

(c) errors in the method which results in missing data (for example, areas on a biotechnology array from which data is missing).

The inventors have developed a method for analysis of data generated from systems which preferably permits identification of components of the system which exhibit a response pattern under a test condition.

### DESCRIPTION OF THE INVENTION

In a first aspect, the invention provides a method for identifying components of a system from data generated from the system, which components exhibit a response pattern associated with a test condition applied to the system, comprising the steps of:

(a) specifying design factors to specify the type of response pattern for the test condition;

(b) identifying a linear combination of components from the input data which correlate with the response pattern.

Preferably, the method includes the step of defining a matrix of design factors.

The inventors have developed a method whereby linear combinations of components from a system can be computed from large amounts of data whereby the linear combination of components fits or correlates with a specified response pattern. Thus, using this method, specific patterns in the data can be searched for and components exhibiting this pattern identified. This facilitates rapid screening of the data from a system for significant components.

The linear combination of components is preferably of the form:

y=a₁X₁+a₂X₂+a₃X₃ . . . aₙXₙ

Wherein y is the linear combination a₁-aₙ are component weights and X₁-Xₙ are data values generated from the method applied to the system for components of the system.

Preferably, a linear combination of components is chosen such that a linear regression of the linear combination of components on the design factors has as much predictive power as possible. The component weights are assessed in a manner such that the values of the component weights for components which do not correlate with the design factors are eliminated from the linear combination.

The method of the present invention has the advantage that it requires usage of less computer memory than prior art methods. Accordingly, the method of the present invention can preferably be performed rapidly on computers such as, for example, laptop machines. By using less memory, the method of the present invention also allows the method to be performed more quickly than prior art methods for analysis of, for example, biological data.

The method of the present invention is suitable for use in the analysis of any system in which components which exhibit a response pattern are sought. Suitable systems include, for example, chemical systems, biological systems, geological systems, process monitoring systems and financial systems including, for example, credit systems, insurance systems, marketing systems or company record systems.

The method of the present invention is particularly suitable for use in the analysis of results obtained from methods applied to biological systems.

The data from the system is preferably generated from methods applied to the system. For example, the data may be a measure of a quantity of the components of the system, the presence of components in a system, or any other quantifiable feature of the components of a system.

The data may be generated using any methods for measuring the components of a system. The data may be generated from, for example, biotechnology array analysis such as DNA array analysis, DNA microarray analysis (see for example, Schena et al., 1995, Science 270: 467-470; Lockhart et al. 1996, Nature Biotechnology 14: 1649; U.S. Pat. No. 5,569,588), RNA array analysis, RNA microarray analysis, DNA microchip analysis, RNA microchip analysis, protein microchip analysis, carbohydrate analysis, antibody array analysis, or analysis such as DNA electrophoresis, RNA electrophoresis, one dimensional or two dimensional protein electrophoresis, proteomics.

The components of the method of the present invention are the components of the system that are being measured. The components may be any measurable component of the system. The components may be, for example, genes, proteins, antibodies, carbohydrates. The components may be measured using methods for detecting the amount of, for example, genes or portions thereof, DNA sequences such as oligonucleotides or cDNA, RNA sequences, peptides, proteins, carbohydrate molecules or any other molecules that form part of the biological system. For example, in a DNA microarray, the component may be a gene or gene fragment. In an antibody array, the component may be a monoclonal antibody, polyclonal antibody, Fab fragment, or any other molecule that contains an antigen binding site of an antibody molecule.

It will be appreciated by those skilled in the art that, the components need not be known, but merely identifiable in a manner to permit a correlation to be made between a linear combination of the components and the design matrix. For example, each components may have a unique identifier such as an arbitrarily selected number or name.

The response pattern specified by the design factors may be any desired pattern. In one embodiment, the response pattern specified by the design factors is derived from known data. Thus, a response pattern derived from known data will identify response patterns that are significantly similar to a known response pattern. For example, a matrix of design factors may be provided for gene expression that correlates with a known gene expression pattern. For example, a particular expression pattern of a particular yeast gene over a particular growth period.

In another embodiment, the response pattern specified by the design factors is derived from the input array data. In this case, a response pattern derived from the input array data will group components of the array which exhibit significantly similar response patterns.

In yet another embodiment, the response pattern specified by the design factors is selected to identify any arbitrary response pattern.

The test conditions of the method of the invention may be any test conditions applied to a system. For example, in the case of a biological system, the test condition may be the growth conditions (such as temperature, time, growth medium, exposure to one or more test compounds) applied to an organism prior to measurement of the components of the system, the phenotype(such as a tumour cell, benign cell, advanced tumour cell, early tumour cell, normal cell, mutant cell, cell from a particular tissue or location)of an organism prior to measurement of the components of the system.

As discussed above, to identify a linear combination of components from input data, let y^{T}=a^{T}_{X} whereby y is a linear combination in which X is an input data matrix of data, preferably array data, having n rows of components and k columns of test conditions, and a is a matrix of values or weights to be applied to the input data. The significance of regression co-efficients of y on a matrix of design factors T may be determined by the ratio:

$$λ = \frac{( {y}^{T} Py ) / r}{{y}^{T} ( I - P ) y / ( n - r )} \qquad 1$$

Wherein

P=T(T^{T}T)⁻¹T^{T}; and

T is a kxr design matrix;

whereby values of a are selected to maximise λ.

Substituting a^{T}X for y in equation 1 and ignoring the constant divisors provides the following equation:

$$λ = \frac{{a}^{T} {XPX}^{T} a}{{a}^{T} X ( I - P ) {X}^{T} a} \qquad 2$$

Thus, a linear combination of components ã may be computed by finding the maximum value of λ in equation 2. However, there are linear combinations (ã) for which the denominator of equation 2 is zero and therefore λ is infinite. Thus, in one embodiment, the present invention provides algorithms for determining a whereby a^{T}X(I−P)X^{T}a is not zero.

In one embodiment, the linear combination is computed by solving the generalised eigenvalue problem of:

(XPX^{T}−λX(I−P)X^{T})ã=0 3

for λ and ã

wherein X is a data matrix having n rows of components and k columns of test conditions and

P=T(T^{T}T)⁻¹T^{T} wherein T is a matrix of k rows of design factors and r columns.

Equation 3 may be solved by the following algorithm:

Let B=XPX^{T} and W=X(I−P)X^{T}

Then to maximise the ratio (equation 2) in the case that W is non-singular we would solve

(B−λW)ã=0 4

One approach for doing this is to rewrite equation 4 as

$$( \begin{matrix}{W}^{\frac{1}{2}} {BW}^{\frac{1}{2}} - λ I ) {W}^{\frac{1}{2}} a = 0 & \end{matrix} \qquad 5$$

and solve this eigen equation.

If

$${W}^{\frac{1}{2}}$$

in equation 5 is replaced in the singular case by

$${W}^{\frac{1}{2}} = U [ \begin{matrix}{Δ}_{1}^{\frac{1}{2}} & 0 \\ 0 & 0\end{matrix} ] {U}^{T} \qquad 6$$

where Δ₁ is the diagonal matrix of ‘non zero’ eigen values of W it is easy to see that equation 5 becomes

$$( [ \begin{matrix}{Δ}_{1}^{\frac{1}{2}} {U}_{1}^{T} {BU}_{1} {{Δ}_{1}}^{\frac{1}{2}} & 0 \\ 0 & 0\end{matrix} ] - λ I ) [ \frac{{Δ}_{1}^{\frac{1}{2}} {U}_{1}^{T} \underline{a}}{0} ] = 0 \qquad 7$$

where U=[U₁U₂] is partitioned conformable with Δ₁. Maximising equation 2 subject to a=U₁ã (i.e a is constrained to be in the range space of W) gives rise to the eigen equation defined by the top left hand block of the lefthand side of equation 7.

Equation 4 may be solved directly without requiring calculation of XPX^{T} or X(I−P)X^{T} using the generalised singular value decomposition, see Golub and Van Loan (1989), Matrix Computations, 2^{nd} Ed. Johns Hopkins University Press, Baltimore.

Alternatively, X(I−P)X^{T} in equation 3 may be replaced with X(I−P)X^{T}+σ²I. Thus, in another embodiment, the linear combination may be identified by solving the equation:

(XPX^{T}−λX(I−P)X^{T}+σ²I)ã=0 for λ and a 8

wherein X is a data matrix having n rows of components and k columns of test conditions; and

P=T(T^{T}T)⁻¹T^{T} wherein T is a matrix of k rows of design factors and r columns and a is a weight matrix for the linear combination y^{T}=ã^{T}X.

In a preferred embodiment, the invention provides a method for identifying components of a system from data generated from the system, which exhibit a response pattern associated with a set of test conditions applied to the system, comprising the steps of:

(a) specifying design factors to specify the type of response patterns for the test conditions;

(b) formulating a model for the residuals of a regression of the input data on the design factors;

(c) estimating parameters for the model;

(d) computing a linear combination of components using the model and its estimated parameters.

Preferably, the method includes the step of defining a matrix of design factors.

Preferably, the system is a biological system. Preferably, the data generated from a method applied to the system is generated from a biotechnology array.

The inventors have found that the denominator of equation 2 may be replaced with the quantity a^{T}Va wherein V is the covariance matrix of the residuals from the regression model. Thus in one embodiment, the linear combination may be computed by maximising the ratio:

$$λ = \frac{{a}^{T} {XPX}^{T} a}{{a}^{T} Va} \qquad 9$$

Equation 9 may be used to give the following optimal a:

a=λ^{-1/2}XPu 10

wherein a is a weight matrix for the linear combination

y=a^{T}X,

P=T(T^{T}T)⁻¹T^{T},

u is an eigenvector of P(XV⁻¹X^{T})P or equivalently a left singular vector of V^{-1/2}XP;

and X is an nxk data matrix from data generated from a method applied to the system, the data being from n components and k test conditions.

This approach has the advantage that the method of the invention does not require storage of matrices larger than nxk. Thus, an advantage of the method of the invention is that it permits analysis of data obtained from large numbers of components or large amounts of components and test conditions.

In a preferred embodiment, the covariance matrix V is replaced by its maximum likelihood estimator. Maximum likelihood estimates are obtained from a model for the microarray data. In this preferred embodiment, the data are modelled by a normal distribution, which is completely specified by the mean and variance.

The model of the method of the present invention may comprise a mean model and a variance model. The mean model may be defined by the equation:

E{X^{T}}=TB^{T}

wherein X is an nxk matrix of data, preferably array data, having n rows of components and k columns of test conditions, T is a kxr matrix of design factors having k rows and r columns and B is an nxr matrix of regression parameters.

The variance model may be defined by the equation:

V ar{vec{X^{T}}}=Iₖ\mathring{x}V 12

where V is a covariance matrix:

V=ΛΦΛ^{T}+σ²I,Λₙₓₛ

with constraints

Φₛₓₛ diagonal and Λ^{T}Λ=I.

The variance model and mean model together determine the likelihood. From (11) and (12) we may write twice the negative log likelihood as:

L=klog|V|+tr{(X¹−TB¹)V⁻¹(X−BT¹)} 13

The parameters to be estimated in the model include Λ, Φ, σ² and the regression coefficient B. In one embodiment, an estimate of regression coefficients B for the mean model is computed using standard least squares:

\hat{B}=X^{T}T(T^{T}T)⁻¹

Substituting into Equation 13 we obtain the likelihood of V conditional on B=\hat{B}:

L=L(\hat{B})=klog|V|+tr{V⁻¹RR^{T}}

where R=X−\hat{B}T^{T}

In one embodiment, the parameters for the covariance matrix are estimated by computing the maximum likelihood estimates (MLE) for the covariance matrix, conditional on the regression parameters. The covariance matrix of the variance model may be defined by the equation:

V=ΛΦΛ^{T}+σ²I 14

To find the maximum likelihood estimate (MLE) of the parameters of V, we proceed as follows:

$$\begin{matrix}From V = {ΛΦΛ}^{T} + {σ}^{2} I we get & \\ V = {[ {ΛΛ}^{*} ] [ \begin{matrix}Φ + {σ}^{2} {I}_{s} & 0 \\ 0 & {σ}^{2} {I}_{n - s}\end{matrix} ] [ {ΛΛ}^{*} ]}^{T} & 15\end{matrix}$$

where Λ* is an orthonormal completion of Λ. It may be shown that

$${V}^{- 1} = {[ {ΛΛ}^{*} ] [ \begin{matrix}{( Φ + {σ}^{2} {I}_{s} )}^{- 1} & 0 \\ 0 & {σ}^{- 2} {I}_{n - s}\end{matrix} ] [ {ΛΛ}^{*} ]}^{T} \\ = {Λ ( Φ + {σ}^{2} {I}_{s} )}^{- 1} Λ + {σ}^{2} ( I - {ΛΛ}^{T} ) . \qquad 16$$

Hence:

$$\begin{matrix}\vert V \vert = \vert Φ + {σ}^{2} {I}_{s} \vert {( {σ}^{2} )}^{n - s} & \\ = {\prod }_{i = 1}^{s} ( {Φ}_{ii} + {σ}^{2} ) {( {σ}^{2} )}^{n - s} & \\ so & \\ k log \vert V \vert = k \{ {\sum }_{i = 1}^{s} log ( {Φ}_{ii} + {σ}^{2} ) + ( n - s ) log {σ}^{2} \} & 17\end{matrix}$$

Further, we may write:

tr{V⁻¹RR^{T}}=tr{(Φ+σ²Iₛ)⁻¹Λ^{T}RR^{T}Λ}+σ⁻²tr{RR^{T}−Λ^{T}RR^{T}Λ} 18

Combining equation 17 and equation 18, the log likelihood function for Λ, Φ and σ² conditional on B may be obtained. We proceed to maximise this as a function of A subject to the constraint Λ^{T}Λ=I. Forming the Lagrangian and differentiating this with respect to Λ we obtain the equation ∂L/∂Λ=0 where

$$\frac{\partial L}{\partial Λ} = \frac{\partial }{\partial Λ} tr \{ [ {( Φ + {σ}^{2} {I}_{s} )}^{- 1} - {σ}^{- 2} {I}_{s} ] {Λ}^{T} {RR}^{T} Λ \} + tr \{ L ( {Λ}^{T} Λ - I ) \} \qquad 19$$

and L is a lower triangular matrix of Lagrange multipliers. Evaluating this and incorporating the constraint gives

RR^{T}ΛD+ΛL^{T}=0

with Λ^{T}Λ=I

The first equation can be written as

RR^{T}Λ+ΛL^{T}D⁻¹=0 20

where D=(Φ+σ²Iₛ)⁻¹−σ⁻²Iₛ. Note that D is invertible provided all Φᵢᵢ>0.

In one embodiment, the maximum likelihood estimate of σ is computed from the equation:

$${\hat{σ}}^{2} = \frac{1}{k ( n - s )} \{ tr \{ {RR}^{T} \} - {\sum }_{i = 1}^{s} {δ}_{ii} \} \qquad 21$$

wherein s is the number of latent factors in the variance model.

In one embodiment, the maximum likelihood estimate of Φ is computed from the equation:

\hat{Φ}_{ii+\hat{σ}}²=δᵢᵢ/k 22

In one embodiment, δ is defined by the equation:

δᵢᵢ=(Λᵢ^{T}RR^{T}Λᵢ) 23

wherein δᵢᵢ is the i^{th} eigenvalue of RR^{T}.

Equations

$${\hat{σ}}^{2} = \frac{1}{k ( n - s )} \{ tr \{ {RR}^{T} \} - {\sum }_{i = 1}^{s} {δ}_{ii} \} , \qquad ( 21 )$$

\hat{Φ}ᵢᵢ+\hat{σ}²=δᵢᵢ/k (22), and δᵢᵢ=(Λᵢ^{T}RR^{T}Λᵢ) (23) are derived as follows:

Premultiplying RR^{T}ΛD+ΛL^{T}=0 by Λ^{T} and using Λ^{T}Λ=I shows that L is symmetric and hence diagonal. It follows that the columns of A are eigenvectors of RR^{T}.

Similarly we obtain

$$\frac{\partial L}{\partial {Φ}_{ii}} = \frac{k}{( {Φ}_{ii} + {σ}^{2} )} - \frac{{δ}_{ii}}{{( {Φ}_{ii} + {σ}^{2} )}^{2}}$$

$$\frac{\partial L}{\partial {σ}^{2}} = {\sum }_{i = 1}^{s} \frac{k}{( {Φ}_{ii} + {σ}^{2} )} + \frac{k ( n - s )}{{σ}^{2}} - {\sum }_{i = 1}^{s} \frac{{δ}_{ii}}{{( {Φ}_{ii} + {σ}^{2} )}^{2}} - \frac{1}{{( {σ}^{2} )}^{2}} \{ tr \{ {RR}^{T} \} - {\sum }_{i = 1}^{s} {δ}_{ii} \}$$

where δᵢᵢ=(Λᵢ^{T}RR^{T}Λᵢ) is the i^{th} eigenvalue of RR^{T}.

It follows that

$${\hat{Φ}}_{ii} + {\hat{σ}}^{2} = {δ}_{ii} / k$$

$${\hat{σ}}^{2} = \frac{1}{k ( n - s )} \{ tr \{ {RR}^{T} \} - {\sum }_{i = 1}^{s} {δ}_{ii} \}$$

The number of latent factors in the model for the covariance matrix may be estimated by performing likelihood ratio tests, cross validation tests or Bayesian procedures. In one embodiment, the number of factors in the variance model is determined by performing a series of likelihood ratio tests, for increasing numbers of factors. The number of factors is chosen such that the test for further increase in the number of factors is not statistically significant. The likelihood ratio test statistic is computed using the equation:

$$- 2 log L = k \{ {\sum }_{i = 1}^{s} log ( {δ}_{ii} / k ) + ( n - s ) log \{ {\sum }_{s + 1}^{t} {δ}_{ii} / ( k ( n - s ) ) \} \} + kn \qquad 24$$

and the number of parameters is ns+s+1−s(s+1)/2.

In a preferred embodiment, the number of factors, s, in the variance model is determined by performing a Bayesian method, preferably based on a method for selecting the number of principle components given in Minka T. P. 2000, Automatic choice of dimensionality for PCA, MIT Media Laboratory Perceptual Computing Section Technical Report No. 514 (Minka (2000)). We note that the problem of choosing basis functions in the factor analysis model i.e. the number of left singular vectors in an singular value decomposition (SVD) of the residual matrix to include can be thought of as the problem of selecting the number of right singular vectors or principal components. Writing λᵢ for the eigenvalues of R^{T}R, in Minka(2000) the number of principal components is chosen to maximise

$$log P ( R \vert s ) = log P ( u ) - 0.5 n {\sum }_{j = 1}^{s} log ( {λ}_{j} ) - 0.5 n ( k - s ) log ( v ) + 0.5 ( m + s ) log ( 2 π ) - 0.5 log det ( {A}_{z} ) - 0.5 s log ( n )$$

where m=ks−s(s+1)/2,

$$log P ( u ) = - s log ( 2 ) + {\sum }_{i = 1}^{s} log ( Γ ( ( k - i + 1 ) / 2 ) ) - 0.5 ( k - i + 1 ) log ( π )$$

$$v = ( {\sum }_{j = s + 1}^{k} {λ}_{j} ) / ( k - s )$$

$$and$$

$$log det ( {A}_{z} ) = {\sum }_{i = 1}^{s} {\sum }_{j = i + 1}^{k} log ( ( {\hat{λ}}_{j}^{- 1} - {\hat{λ}}_{i}^{- 1} ) ( {λ}_{i} - {λ}_{j} ) n )$$

$$where$$

$${\hat{λ}}_{j} = \{ \begin{matrix}{λ}_{j} , for j \leq k \\ v , otherwise .\end{matrix}$$

More reliable results are obtained using the Bayesian approach if it is used on a subset of the genes, chosen to show high correlation with the response pattern specified by the design factors.

The present invention also provides a means to determine the shape of the relationship between the linear combination of components and the response pattern specified by the design factors. The inner product of the linear combinations with the data matrix results ih a loading for each array. These loadings may be plotted against the columns of the design factors to reveal the shape of the response.

The present invention also provides for testing the significance of the components of a linear combination, and/or the overall strength of the relationship between the linear combination and the design factors. In one embodiment, the method comprises the further steps of:

(a) determining the significanceof each weight of the linear combination; and

(b) setting non-significant weights to zero.

In a preferred embodiment, the significance of the weights of the linear combination is determined by a permutation test comprising the steps of:

(a) randomising the data, preferably biotechnology array data, within each row;

(b) Computing the weights and eigenvalues from the randomised data;

(c) repeating steps (a) and (b) a plurality of times; and

(d) determining a distribution for the weights and eigenvalues computed from the randomised data;

(e) determining the position of weights and eigenvalues computed from non-randomised data, preferably biotechnology array data, relative to the distribution of the weights and eigenvalues computed from randomised data;

(f) estimating the significance of each weight computed from the non-randomised data.

In a preferred embodiment, the significance of the relationship between the linear combinations of components and the response pattern specified by the design factors may be determined in an analogous way. For each randomisation step (a) above, the loadings are formed as inner products of the linear combinations with the data matrix. The multiple correlation between these loadings and the response pattern specified by the design factors is calculated. The significance of the overall relationship is evaluated by determining the position of the multiple correlation coefficient from non-randomised data with the distribution of the multiple correlation coefficient calculated from randomised data.

The present invention also provides methods for estimating missing values from the data. In one embodiment, missing values are estimated using an EM algorithm. In a preferred embodiment, the method comprises estimating missing data values of array data by:

(a) estimating initial values of B, Γ, Φ, σ² by replacing missing values with simple estimates and calculating maximum likelihood estimates assuming the data was complete;

(b) Computing E{X|o₁, . . . oₖ}, E{RR^{T}|o₁, . . . oₖ} the expected values of the data array and the residual matrix under the model given the observed data (where oᵢ is defined below);

(c) Substitute quantities for (b) into likelihood equations assuming complete data to obtain new estimates of B, Γ, φ and σ²;

(d) Repeat steps (b) to (d) until convergence.

In one embodiment, the EM algorithm is performed as follows:

From equations 18 and 20:

R=X−BT^{T},V=ΛΦΛ^{T}+σ²I

For the ith column of R, Rᵢ say, we can partition Rᵢ as

$${R}_{i} = [ \begin{matrix}{o}_{i} \\ {u}_{i}\end{matrix} ] , V = [ \begin{matrix}{V}_{oo} & {V}_{ou} \\ {V}_{uo} & {V}_{uu}\end{matrix} ] , {V}^{- 1} = [ \begin{matrix}{V}^{oo} & {V}^{ou} \\ {V}^{ou} & {V}^{uu}\end{matrix} ] \qquad 25$$

where oᵢ denotes the observed residual component and uᵢ denotes the missing residual component. To do the E step of the EM algorithm we need to compute the expected values

E{Rᵢ|oᵢ} and E{RᵢRᵢ^{T}|oᵢ} 36

Note that we are also conditioning on a set of parameter values, B, Λ, Φ and σ², however for easy of presentation we do not represent this in the following.

It can be shown that

$$\begin{matrix}E \{ {u}_{i} \vert {o}_{i} \} = {{V}_{u 0} ( {V}_{00} )}^{- 1} {o}_{i} \\ = - {( {V}^{uu} )}^{- 1} {V}^{uo} {o}_{i} \\ = {Co}_{i} ( say )\end{matrix} \\ Hence \\ E \{ {R}_{i} \vert {o}_{i} \} = [ \begin{matrix}I \\ C\end{matrix} ] {o}_{i} \qquad 27$$

From the definition of R we obtain

$$E ( {X}_{i} \vert {o}_{i} ) = [ \begin{matrix}I \\ C\end{matrix} ] {o}_{i} + {BT}^{T} {e}_{i} \qquad 28$$

where eᵢ is a kxl vector with zeros except in the ith position which is a one.

Now writing V^{uu} for V^{u}^{ᵢ}^{u}^{ᵢ} we have

Let

$$\begin{matrix}E \{ {R}_{i} {R}_{i}^{T} \vert {o}_{i} \} = [ \begin{matrix}I & 0 \\ C & I\end{matrix} ] [ \begin{matrix}{o}_{i} {o}_{i}^{T} & 0 \\ 0 & {( {V}^{uu} )}^{- 1}\end{matrix} ] [ \begin{matrix}I & {C}^{T} \\ 0 & I\end{matrix} ] \\ = [ \begin{matrix}I \\ C\end{matrix} ] {o}_{i} {o}_{i}^{T} [ {IC}^{T} ] + [ \begin{matrix}0 & 0 \\ 0 & {( {V}^{uu} )}^{- 1}\end{matrix} ] \\ = {R}_{i}^{*} {R}_{i}^{T} + [ \begin{matrix}0 \\ {L}_{i}\end{matrix} ] [ 0 {L}_{i}^{T} ]\end{matrix} \\ Where {( {V}^{uu} )}^{- 1} = {L}_{i} {L}_{i}^{T} . \qquad 29$$

It follows that

$$E [ {RR}^{T} \vert {o}_{i} … {o}_{k} ] = {\sum }_{i = 1}^{k} {R}_{i}^{*} {R}_{i}^{T} + {\sum }_{i = 1}^{k} {S}_{i} {S}_{i}^{T} \qquad 30$$

where

$${S}_{i} = {P}_{i}^{T} [ \begin{matrix}0 \\ {L}_{i}\end{matrix} ]$$

is nxmᵢ. Here mᵢ is the number of missing values in column i and Pᵢ is a permutation matrix with the property that

$${P}_{i} {R}_{i} = [ \begin{matrix}{o}_{i} \\ {u}_{i}\end{matrix} ] .$$

$$\begin{matrix}Define & \\ m = {\sum }_{i} {m}_{i} and \hat{R} = [ {R}_{1}^{*} {…R}_{k}^{*} {…⋮S}_{1} {…S}_{k} ] , nx ( k + m ) \\ then \\ E \{ {RR}^{T} \vert {o}_{i} , … {o}_{k} \} = \hat{R} {\hat{R}}^{T} & 31\end{matrix}$$

A similar expression also follows from writing

$${\sum }_{i} [ \begin{matrix}0 & 0 \\ 0 & {( {V}^{{u}_{i} {u}_{i}} )}^{- 1}\end{matrix} ] = [ \begin{matrix}0 & 0 \\ 0 & D\end{matrix} ] = [ \begin{matrix}0 & 0 \\ 0 & {LL}^{T}\end{matrix} ] \qquad 32$$

This requires only 1 (larger) matrix factorisation and the dimension of D may be much less than m if common genes are missing (across columns of X).

The above expressions enable the computation of maximum likelihood estimates by using the SVD of R, thus saving on storage requirements.

From equations 35 and 36 it can be seen that the matrix inversion (V^{uu})⁻¹ is required. This may be a large matrix if there are many missing values in a column of R. In such cases we note the following:

V^{uu}=Λᵤ(Φₛ+σ²Iₛ)⁻¹Λᵤ^{T}+σ⁻²(I−ΛᵤΛᵤ^{T}) 33

where Λᵤ denotes an appropriate subset of rows of Λ (Λᵤ is mxs).

V^{uu} can be rewritten as

Λᵤ{(Φₛ+σ²Iₛ)⁻¹−σ²Iₛ}Λᵤ^{T}+σ⁻²I 34

Hence using the formula

(A+BDB^{T})⁻¹=A⁻¹−A⁻¹B(B^{T}A⁻¹B+D⁻¹)⁻¹B^{T}A⁻¹ 35

it can be shown that

(V^{uu})⁻¹=σ²I−σ²Λᵤ(σ²Λᵤ^{T}Λᵤ+{(Φₛ+σ²Iₛ)⁻¹−σ⁻²I_{s}}⁻¹ )⁻¹Λᵤ^{σ}^{²} 36

Note that this only requires the inverse of an s×s matrix where s is the number of basis functions in the variance model and is independent of m.

The EM algorithm discussed above requires the factorisation of the matrices V^{uu} which may be reasonably large if there are substantial numbers of missing values. An alternative algorithm which does not require this is as follows:

$$\begin{matrix}Write & \\ {R}_{i} = {X}_{i} - {BT}^{T} {e}_{i} and {R}_{i} = [ \begin{matrix}{o}_{i} \\ {u}_{i}\end{matrix} ] for i = 1 , … , k . & 37\end{matrix}$$

Then assuming normality, we can write the log likelihood of the data as:

$$L = log L = {\sum }_{i = 1}^{k} log f ( {u}_{i} \vert {o}_{i} θ ) + log g ( {o}_{i} \vert {o}_{i} θ ) \qquad 38$$

where f is the conditional normally density function of uᵢ given oᵢ and g is the marginal density function of oᵢ. The vector of parameters θ is B, Λ, φ and σ².

Now writing L=L(u₁, u₂, . . . , uₖ, σ), an iterative algorithm can be specified for maximising equation 45 as follows:

(a) Specify initial values θₒ

(b) For iteration n>0 maximise L as a function of u₁, . . . , uₖ. From the form of 45 we can do this independently for each uᵢ and since logf (uᵢ|oⁱ, θₙ) is a (conditional) normal distribution the maximum occurs at ûᵢ⁽ⁿ⁾=E{uᵢ|o_{l, θ}ₙ}. This of course is a calculation done in the E step of the original E-M algorithm.

(c) With uᵢ=ûᵢ⁽ⁿ⁾ for i=1, . . . ,k maximise 45 as a function of θ ignoring the dependence of uᵢ on θ (i.e treating the uᵢ as now fixed) to produce θₙ₊₁

(d) Go to 2 until some stopping criteria is satisfied.

The above algorithm preferably produces a sequence with the property that for n≧0

L(ũ⁽ⁿ⁺¹⁾, θₙ₊₁)≧L(ũ⁽ⁿ⁾, θₙ) 39

where ũ⁽ⁿ⁾=(uᵢ⁽ⁿ⁾, . . . , uₖ⁽ⁿ⁾).

Step (c) of the algorithm corresponds to ignoring the V^{uu} terms in the calculation of E{RR^{T}|σ₁, . . . , oₖ} of the EM algorithm, and then doing the M step of the EM algorithm. (Note that the estimation of B can be done independently of the other parameters in θ.)

We can completely remove the need to calculate (V^{uu})⁻¹ in step (b) of the above algorithm by noting that we can use a cyclic ascent algorithm to maximise log f(uᵢ|oᵢ, θ) as follows:

Let the components of uᵢ be (uⱼᵢ, j=1, . . . mᵢ)

Maximising over uᵢᵢ (say) with u₋ₗᵢ=(uⱼᵢ, j≠1) fixed, corresponds to computing E{uₗᵢ|u₋ₗᵢ, oᵢ, θ}

To see this write:

logf(uᵢ|oᵢθ)=logf(uₗᵢ|u₋ₗᵢ, oᵢ, θ)+logh(u₋ₗᵢ|oᵢ, θ) 40

where h is a conditional normal density. Now note that the first term in equation 15 has a maximum at E{uₗᵢ|u₋ₗᵢ, oᵢ, θ} and this can be computed purely from the elements of V⁻¹ given earlier.

Iterating over l=1 . . . , mᵢ will produce the (unique) maximum of logf(uᵢ|oᵢ, θ) namely E{uᵢ|oᵢ, θ}.

This method requires only one matrix factorisation and therefore reduces storage requirements. In a preferred embodiment, the missing values are estimated at the same time that parameters for the model are estimated.

The identification method of the present invention may be implemented by appropriate computing systems which may include computer software and hardware.

In accordance with a second aspect of the present invention, there is provided a computer program which includes instructions arranged to control a computing device to identify linear combinations of components from input data which correlate with a response pattern defined by a matrix of design factors specifying types of response patterns for a set of test conditions in a system.

The computer program may implement any of the preferred algorithms and method steps of the first aspect of the present invention which are discussed above.

In accordance with a third aspect of the present invention, there is provided a computer readable medium providing a computer program in accordance with the second aspect of the present invention.

In accordance with a fourth aspect of the present invention, there is provided acomputer program, including instructions arranged to control a computing device, in a method of identifying components from a system which exhibit a pre-selected response pattern to test conditions applied to the system, and wherein a matrix of design factors specifying the response patterns for the test conditions is defined, to formulate a module for the residuals of a regression of the input array data on the design factors, to estimate parameters for the model and compute a linear combination of components using the model and the estimated parameters.

The computer program may be arranged to implement any of the preferred method and calculation steps discussed above in relation to the second aspect of the present invention.

In accordance with a fifth aspect of the present invention, there is provided a computer readable medium providing a computer program in accordance with the fourth aspect of the present invention.

In accordance with a sixth aspect of the present invention there is provided an apparatus for identifying components from a system which exhibit a response pattern(s) associated with test conditions applied to the system, and wherein a matrix of design factors to specify the type of response patterns for the set of tests and conditions is defined, the apparatus including a calculation device for identifying linear combinations of components from the input data which correlate with the response pattern.

In accordance with an seventh aspect of the present invention, there is provided an apparatus for identifying components from a system which exhibit a preselected response pattern to a set of test conditions applied to the system, wherein a matrix of design factors to specify the response pattern(s) for the test conditions is defined, the apparatus including a means for formulating a model for the residuals of a regression of the input array data on the design factors, means for estimating parameters for the model and means for computing a linear combination of components using the model and the estimated parameters.

A computing system including means for identifying components including means for implementing any of the preferred algorithms and method steps of the first aspect of the present invention which are discussed above.

Where aspects of the present invention are implemented by way of a computing device, it will be appreciated that any appropriate computer hardware e.g. a PC or a mainframe or a networked computing infrastructure, may be used.

### BRIEF DESCRIPTION OF THE FIGURES

FIG. 1 shows a graphical plot of a matrix of design factors of a preferred method of the invention (top) and gene expression patterns of the genes of yeast from microarray data that correlate to the response pattern specified by those design factors (bottom). The x-axis is the time of growth of the yeast at which gene expression was measured. The y-axis is the value design factor given for each time (top) or the level of gene expression (bottom).

FIG. 2 shows agraphical plot of a matrix of design factors of a preferred method of the invention (top) and gene expression patterns of the genes of yeast from microarray data that correlate to the response pattern specified by the design factors (bottom). The x-axis is the time of growth of the yeast at which gene expression was measured. The y-axis is the value design factor given for each time (top) or the level of gene expression (bottom).

FIG. 3 shows a graphical plot of a matrix of design factors of a preferred method of the invention (top) and gene expression patterns of the genes of yeast from microarray data that correlate to the response pattern specified by the design factors (bottom). The x-axis is the time of growth of the yeast at which gene expression was measured. The y-axis is the value design factor given for each time (top) or the level of gene expression (bottom).

FIG. 4 shows a graphical plot of a matrix of design factors of a preferred method of the invention (top) and gene expression patterns of the genes of GC B-like diffuse large B cell lymphoma and activated B-like diffuse large B cell lymphoma from microarray data that correlate to the response pattern specified by the design factors (bottom). The x-axis is the class of lymphoma. The y-axis is the value design factor given for each class (top) or the level of gene expression (bottom).

FIG. 5 shows a graphical plot of a matrix of design factors of a preferred method of the invention (top) and gene expression patterns of the genes of yeast from the microarray data listed in table 1 that correlate to the response pattern specified by those design factors (bottom). The x-axis is the time of growth of the yeast at which gene expression was measured. The y-axis is the value design factor given for each time (top) or the level of gene expression (bottom).

FIG. 6 shows a graphical plot of a matrix of design factors of a preferred method of the invention (top) and gene expression patterns of the genes of yeast from the microarray data listed in table 1 that correlate to the response pattern specified by the design factors (bottom). The x-axis is the time of growth of the yeast at which gene expression was measured. The y-axis is the value design factor given for each time (top) or the level of gene expression (bottom).

FIG. 7 shows a graphical plot of a matrix of design factors of a preferred method of the invention (top) and gene expression patterns of the genes of yeast from the microarray data listed in table 1 that correlate to the response pattern specified by the design factors (bottom). The x-axis is the time of growth of the yeast at which gene expression was measured. The y-axis is the value design factor given for each time (top) or the level of gene expression (bottom).

FIG. 8 shows a graphical plot of a matrix of design factors of a preferred method of the invention (top) and gene expression patterns of the genes of GC B-like diffuse large B cell lymphoma (GC) and activated B-like diffuse large B cell lymphoma (activate) from the microarray data listed in table 2 that correlate to the response pattern specified by the design factors (bottom). The x-axis is the class of lymphoma (GC or activated). The y-axis is the value design factor given for each class (top) or the level of gene expression (bottom)

### EXAMPLES

### Example 1

The data set for this example is the results from a DNA microarray experiment and is reported in Spellman, P. and Sherlock, G., et al. (1998) Comprehensive identification of cell cycle-regulated genes of the yeast Saccharomyces cerevisiae by Microarray Hybridization. Mol. Biol. Cell 9(12):3273-3297.

The data set generated from the microarray experiments described in the above paper can be obtained from the following web site:

http://genome-www4.stnford.edu/MicroArray/SMD/publications.html

The array data consists of n=2467 genes and k=18 samples (times). The matrix of design facors T (design matrix) has r=6 columns defined by the terms cos(lθ), sin(lθ) for l=1 . . . 3 and θ=(7 mπ)/119, m=0, 1, . . . , 17.

This example illustrates how the method of the present invention can be used to discover sets of genes which exhibit periodic variation within the cell cycle. For this data set, the pattern of periodic variation is a by product of the analysis given the choice of the matrix of design factors T. A search for a priori response pattern could also be specified by choosing r=1 and placing the appropriate pattern in the single column of the design matrix. For this data set we have six canonical vectors a. Note that a=λ^{-1/2}XPu where u is the design factor and a denotes the scores. Two basis functions were used in the factor analysis model. Results for the first three canonicalvariates are given below. The design factor axis is time. Each component has a calculated p value which is highly significant. A list of genes forming a group with a similar pattern of variation over time is given below for the first three canonical vectors. The size of this group can be varied by choosing the significance level applied to the scores (the level here was set at 0.001). Group sizes will tend to be smaller for smaller significance levels.

The results for each canonical vector might be interpreted as implying a similar pattern of variation for each of the three groups but with a phase shift for each group. The low to low cycle period is of the order of 70 minutes which agrees with the results in the paper.

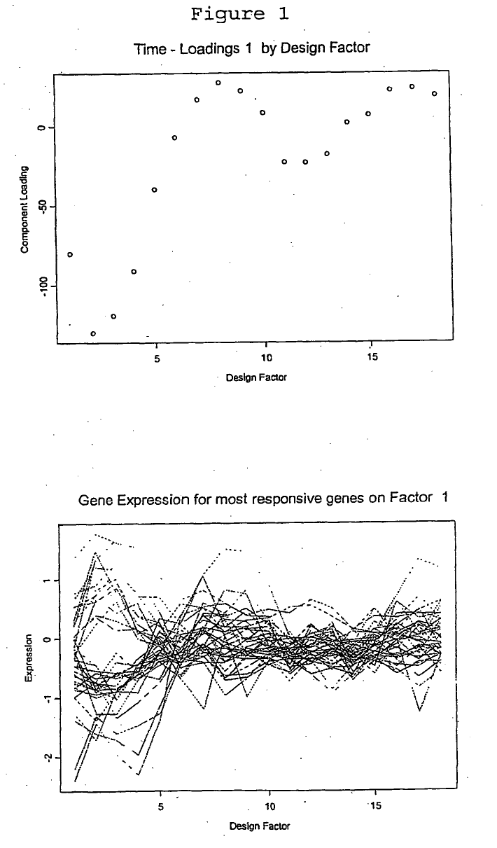

The genes identified are shown below. Results of the gene expression from these genes is shown in FIGS. 1, 2 and 3.

1. Canonical Variatel (see FIG. 1)
d is: 0.9932 p Value is: 0
Spellman Cell Cylcle Data

| Gene | Score | P Value |
|---|---|---|
| YCL040W: | −0.6096 | 0 |
| YPL092W: | −0.4394 | 0 |
| YEL060C: | −0.434 | 0 |
| YDR343C: | −0.4239 | 0 |
| YGR008C: | −0.4047 | 0 |
| YOR347C: | −0.3978 | 0 |
| YLR178C: | −0.3853 | 0 |
| YCL018W: | −0.332 | 0 |
| YMR008C: | −0.3011 | 0 |
| YKL148C: | −0.299 | 0 |
| YGR255C: | −0.2745 | 0 |
| YDR178W: | −0.2454 | 0 |
| YMR152W: | −0.1967 | 0 |
| YMR023C: | −0.1408 | 0 |
| YOL028C: | 0.0956 | 0 |
| YGL244W: | 0.1202 | 0 |
| YIR023W: | 0.1645 | 0 |
| YKL015W: | 0.1809 | 0 |
| YOR330C: | 0.1937 | 0 |
| YPL212C: | 0.2026 | 0 |
| YJL076W: | 0.2201 | 0 |
| YCR034W: | 0.2373 | 0 |
| YFR028C: | 0.2393 | 0 |
| YPL128C: | 0.2482 | 0 |
| YBL170W: | 0.2513 | 0 |
| YBL014C: | 0.2515 | 0 |
| YML123C: | 0.2523 | 0 |
| YGL097W: | 0.2531 | 0 |
| YOR340C: | 0.2677 | 0 |
| YMR274C: | 0.2683 | 0 |
| YFL037W: | 0.2966 | 0 |
| YML065W: | 0.3194 | 0 |
| YOL109W: | 0.3451 | 0 |
| YPR124W: | 0.3752 | 0 |
| YBR142W: | 0.3777 | 0 |
| YBL069W: | 0.4035 | 0 |
| YPL155C: | 0.4282 | 0 |
| YBR243C: | 0.4564 | 0 |
| YLR056W: | 0.4738 | 0 |
| YJR092W: | 0.5137 | 0 |
| YMR058W: | 0.5362 | 0 |
| YGL021W: | 0.6822 | 0 |
| YGR108W: | 0.7574 | 0 |
| YMR001C: | 0.7806 | 0 |
| YBR038W: | 0.8433 | 0 |
| YPR119W: | 1.1639 | 0 |

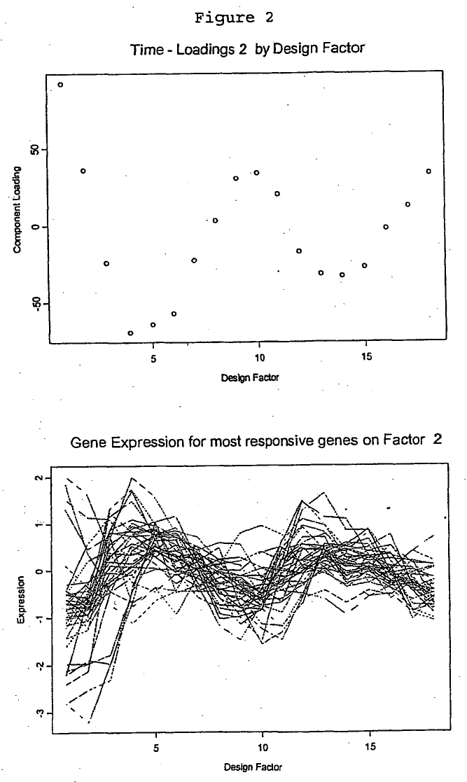

2. Canonical Variate2 (see FIG. 2)
d is: 0.9874 p Value is: 0
Spellman Cell Cycle Data

| Gene | Score | p-Value |
|---|---|---|
| YCL040W | −0.6096 | 0 |
| YBR067C | −0.5403 | 0 |
| YPL092W | −0.4394 | 0 |
| YEL060C | −0.4340 | 0 |
| YDR343C | −0.4239 | 0 |
| YGR008C | −0.4047 | 0 |
| YOR347C | −0.3978 | 0 |
| YLR178C | −0.3853 | 0 |
| YCL018W | −0.3320 | 0 |
| YMR008C | −0.3011 | 0 |
| YKL148C | −0.2990 | 0 |
| YGR255C | −0.2745 | 0 |
| YDR178W | −0.2454 | 0 |
| YMR152W | −0.1967 | 0 |
| YBL079W | 0.1295 | 0 |
| YIR023W | 0.1645 | 0 |
| YKL015W | 0.1809 | 0 |
| YOR330C | 0.1937 | 0 |
| YJL076W | 0.2201 | 0 |
| YNL216W | 0.2330 | 0 |
| YBR222C | 0.2357 | 0 |
| YFR028C | 0.2393 | 0 |
| YPL128C | 0.2482 | 0 |
| YHR170W | 0.2513 | 0 |
| YBL014C | 0.2515 | 0 |
| YGL097W | 0.2531 | 0 |
| YMR274C | 0.2683 | 0 |
| YAL059W | 0.2848 | 0 |
| YBL082C | 0.3054 | 0 |
| YML065W | 0.3194 | 0 |
| YBR142W | 0.3777 | 0 |
| YPL155C | 0.4282 | 0 |
| YBR243C | 0.4564 | 0 |
| YLR056W | 0.4738 | 0 |
| YJR092W | 0.5137 | 0 |
| YGR108W | 0.7574 | 0 |
| YMR001C | 0.7806 | 0 |
| YPR119W | 1.1639 | 0 |

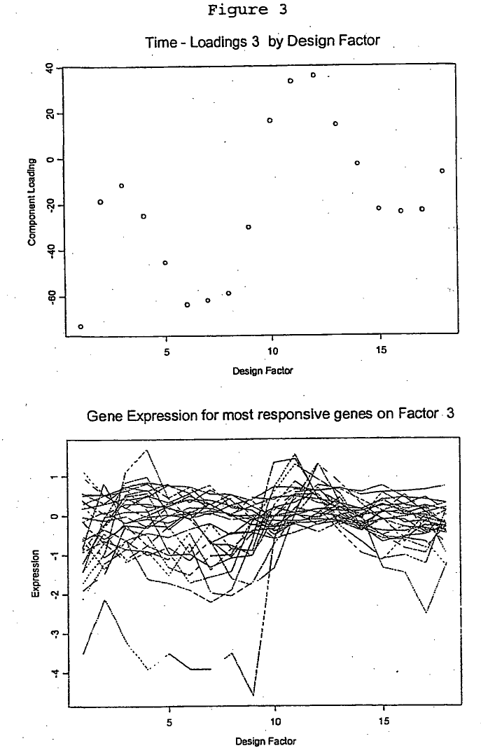

3. Canonical Variate 3 (see FIG. 3)
d is: 0.9773 p Value is: 0.001
Spellman Cell Cylcle Data

| Gene | Score | p-Value |
|---|---|---|
| YKL127W | −0.3295 | 0 |
| YNL280C | −0.3154 | 0 |
| YJL034W | −0.2972 | 0 |
| YCR069W | −0.2856 | 0 |
| YOR079C | −0.2786 | 0 |
| YOR075W | −0.2702 | 0 |
| YOR237W | −0.2587 | 0 |
| YLR299W | −0.2569 | 0 |
| YMR238W | −0.2451 | 0 |
| YOR219C | −0.2103 | 0 |
| YDL207W | −0.2078 | 0 |
| YDL131W | 0.2301 | 0 |
| YNR050C | 0.3180 | 0 |
| YDL182W | 0.3254 | 0 |
| YCR065W | 0.3736 | 0 |
| YGL038C | 0.3944 | 0 |
| YER145C | 0.4387 | 0 |
| YPL256C | 0.6011 | 0 |
| YMR179W | 0.6136 | 0 |
| YPR019W | 0.6201 | 0 |
| YIL009W | 0.6512 | 0 |
| YJL196C | 0.6680 | 0 |
| YDL179W | 0.7498 | 0 |
| YLR079W | 0.7639 | 0 |
| YGR041W | 0.9150 | 0 |
| YJL159W | 0.9385 | 0 |
| YKL185W | 1.1207 | 0 |
| YNL327W | 2.0384 | 0 |

### Example 2

The data set for this example is the results from a DNA microarray experiment and is reported in

Alizadeh, A. A., et al. (2000) Distinct types of diffuse large B-cell lymphoma identified by gene expression profiling. Nature 403:503-511.

The data set generated from the microarray experiments described in the above paper can be obtained from the following web site:

http://genome-www4.stnford.edu/MicroArray/SMD/publications.html

There are n=4026 genes and n=36 samples. In the following DLBCL refers to “Diffuse large B cell Lymphoma”. The samples have been classified into two disease types GC B-like DLBCL (21 samples) and Activated B-like DLBCL (15 samples). The design matrix T has 1 column with values −1 if the sample is in group 2 and +1 if the sample is in group 1. This array data is used to illustrate the potential use of the method of the present invention in discovering genes which are diagnostic of different disease types.

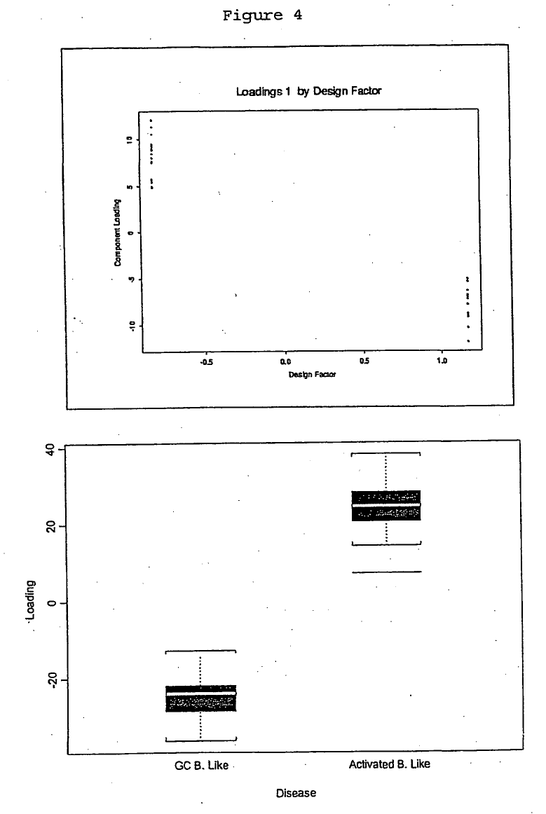

The results of applying the above methodology are given below along with a (partial) list of potentially diagnostic genes. FIG. 4 shows factor loadings calculated for each array, with a Box plot showing the distribution of factor loadings from each disease type. Note the distinct factor loadings for each grouping in the plot.

The genes identified are shown below. Results of the gene expression from these genes is shown in FIG. 4.

Canonical Variatel
d = 0.923 p-value = 0.128

| Gene | Score | p-Value |
|---|---|---|
| GENE3608X | 0.1363 | 0 |
| GENE3326X | 0.1495 | 0 |
| GENE3261X | 0.2013 | 0 |
| GENE3327X | 0.2104 | 0 |
| GENE3330X | 0.2109 | 0 |
| GENE3259X | 0.2217 | 0 |
| GENE3328X | 0.2361 | 0 |
| GENE3329X | 0.2465 | 0 |
| GENE3258X | 0.2534 | 0 |
| GENE1719X | 0.3064 | 0 |
| GENE1720X | 0.3197 | 0 |
| GENE3332X | 0.4509 | 0 |

### Example 3

The data set for this example is listed in Table 1 and is an extract of the data set described in Spellman, P. and Sherlock, G., et al. (1998)

Comprehensive identification of cell cycle-regulated genes of the yeast Saccharomyces cerevisiae by Microarray Hybridization. Mol. Biol. Cell 9(12):3273-3297.

The array data consists of n=100 genes and k=18 samples (times). The matrix of design facors T (design matrix)has r=6 columns defined by the terms cos(lθ), sin(lθ) for l=1 . . . 3 and θ=(7 mπ)/119, m=0, 1, . . . , 17.

This example illustrates how the method of the present invention can be used to discover sets of genes which exhibit periodic variation within the cell cycle. For this data set, the pattern of periodic variation is a by product of the analysis given the choice of the matrix of design factors T. A search for a priori response pattern could also be specified by choosing r=1 and placing the appropriate pattern in the single column of the design matrix. For this data set we have six canonical vectors a. Note that a=λ^{-1/2}XPu where u is the design factor and a denotes the scores. The Bayesian criterion was minimised with 1 basis functions in the factor analysis model. Results for the first three of these are given below. The design factor axis is time. Each component has a calculated p value which is highly significant. A list of genes forming a group with a similar pattern of variation over time is given below for the first three canonical vectors. The size of this group can be varied by choosing the significance level applied to the scores (the level here was set at 0.001). Group sizes will tend to be smaller for higher significance levels.

The results for each canonical vector might be interpreted as implying a similar pattern of variation for each of the three groups but with a phase shift for each group. The low to low cycle period is of the order of 70 minutes which agrees with the results in the paper.

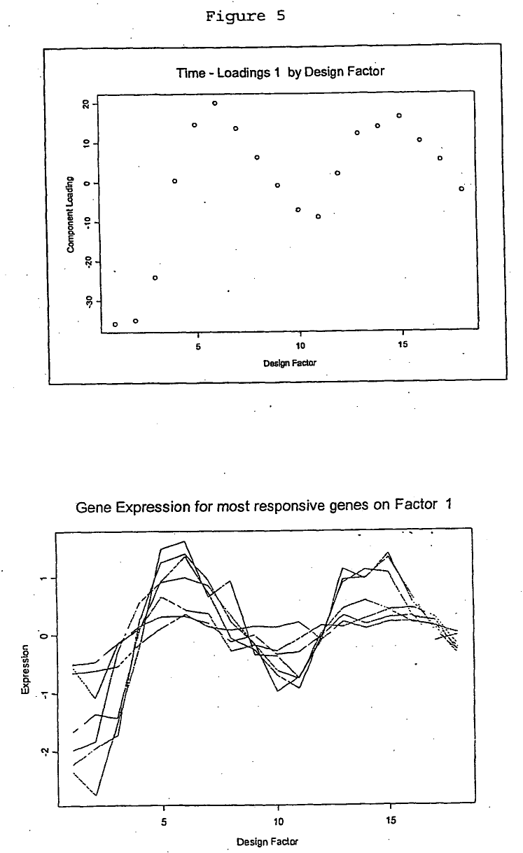

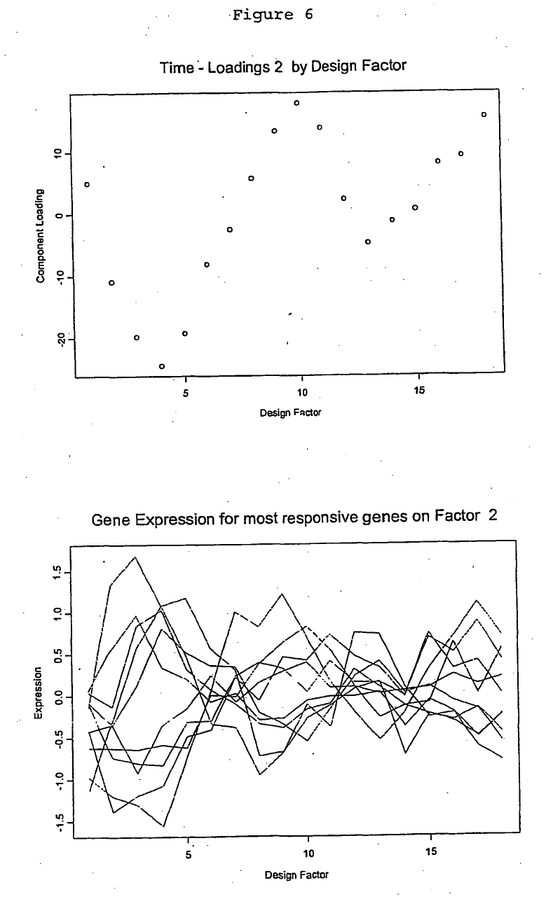

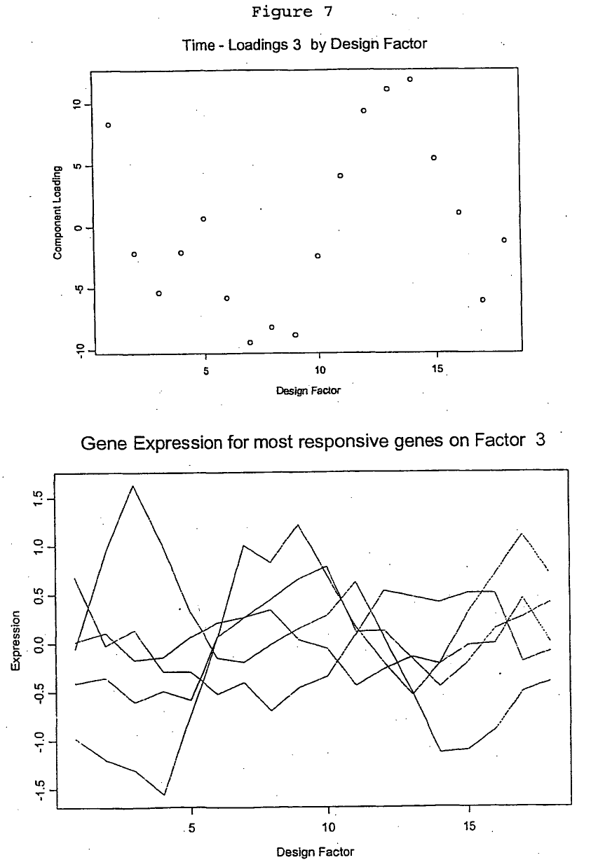

The genes identified are shown below. Results of the gene expression from these genes is shown in FIGS. 5, 6 and 7.

1. Canonical Variatel (see FIG. 1)
d is: 0. p Value is: 0
Spellman Cell Cycle Data

| Gene | Score | p-Value |
|---|---|---|
| YPL092W | −1.0041 | 0.007 |
| YER015W | −0.2681 | 0.008 |
| YGL237C | 0.3235 | 0.009 |
| YKR010C | 0.5801 | 0.000 |
| YNR023W | 0.5849 | 0.001 |
| YCR034W | 0.6459 | 0.000 |
| YAL023C | 0.8632 | 0.000 |
| YBL001C | 0.8943 | 0.001 |
| YPL127C | 1.9008 | 0.000 |
| YNL031C | 2.1047 | 0.000 |
| YNL030W | 2.6658 | 0.000 |
| YBR009C | 2.9482 | 0.000 |
| YPR119W | 0.17948 | 0 |

2. Canonical Variate2 (see FIG. 2)
d is: 0.98320 p Value is: 0
Spellman Cell Cycle Data

| Gene | Score | p-Value |
|---|---|---|
| YOR074C | −1.8064 | 0.000 |
| YIL066C | −1.7692 | 0.000 |
| YCL040W | −1.6460 | 0.000 |
| YJL073W | −1.0510 | 0.000 |
| YOR321W | −0.9528 | 0.000 |
| YKL148C | −0.7819 | 0.000 |
| YDL093W | −0.6411 | 0.007 |
| YJL201W | −0.5744 | 0.009 |
| YOR132W | −0.4864 | 0.009 |
| YKR010C | −0.3184 | 0.009 |
| YFR028C | 0.5224 | 0.006 |
| YKR054C | 0.5821 | 0.007 |
| YNL062C | 0.5910 | 0.005 |
| YHR170W | 0.6916 | 0.000 |
| YNL061W | 0.8039 | 0.001 |
| YLR098C | 1.0517 | 0.001 |
| YOR153W | 1.0690 | 0.001 |
| YOL109W | 1.0760 | 0.000 |
| YAL040C | 1.1198 | 0.000 |
| YGL008C | 1.1682 | 0.002 |
| YMR058W | 1.6489 | 0.000 |
| YMR001C | 2.1982 | 0.000 |

3. Canonical Variate 3 (see FIG. 3)
d is: 0.8870 p Value is: 0.01
Spellman Cell Cycle Data

| Gene | Score | p-Value |
|---|---|---|
| YMR065W | −1.57783303 | 0.000 |
| YJL099W | −0.72894484 | 0.000 |
| YJL044C | 0.515497036 | 0.010 |
| YDR292C | 0.654473229 | 0.010 |
| YIL066C | 1.383495184 | 0.005 |
| YGL038C | 1.617149735 | 0.000 |
| YLR079W | 2.689484257 | 0.000 |
| YKL185W | 3.434889201 | 0.000 |

TABLE 1

| Gene | A1 | A2 | A3 | A4 | A5 | A6 | A7 | A8 | A9 | A10 | A11 | A12 | A13 | A14 | A15 | A16 | A17 | A18 |
|---|---|---|---|---|---|---|---|---|---|---|---|---|---|---|---|---|---|---|
| YAL001C | 0.68 | 0.68 | 0.65 | 0.94 | 0.53 | 0.51 | 0.68 | 1.13 | 0.73 | 0.86 | 0.96 | 1.54 | 0.63 | 0.97 | 0.7 | 1.46 | 0.65 | 1.06 |
| YAL002W | 0.74 | 0.91 | 0.84 | 0.87 | 0.86 | 0.64 | 0.86 | 1.84 | 0.66 | 0.67 | 0.93 | 1.01 | 0.64 | 0.61 | 1.03 | 1.48 | 0.57 | 0.94 |
| YAL023C | 0.51 | 0.30 | 0.74 | 1 | 1.72 | 1.36 | 1.28 | 0.67 | 0.74 | 0.67 | 0.82 | 1.04 | 1.01 | 1.17 | 1.35 | 1.08 | 1.04 | 0.7 |
| YAL040C | 3.71 | 1.57 | 2.1 | 0.47 | 0.7 | 0.66 | 1.45 | 1.11 | 2.23 | 2.59 | 2.16 | 1.07 | 0.93 | 0.73 | 0.96 | 1.01 | 1.46 | 2.01 |
| YBL001C | 0.23 | 0.86 | 0.22 | 0.94 | 1.03 | 1.04 | 1.17 | 1.68 | 0.76 | 0.96 | 0.48 | 0.74 | 1 | 1.06 | 1.08 | 1.11 | 0.82 | 0.8 |
| YBL016W | 7.92 | 1.26 | 0.37 | 0.34 | 0.49 | 0.71 | 0.5 | 2.46 | 0.41 | 0.51 | 0.61 | 0.87 | 0.84 | 0.96 | 0.8 | 1.15 | 0.58 | 1.2 |
| YBR009C | 0.06 | 0.04 | 0.14 | 0.53 | 2.83 | 3.22 | 1.22 | 1.62 | 0.45 | 0.44 | 0.3 | 0.61 | 1.65 | 1.7 | 2.41 | 1.21 | 0.67 | 0.48 |
| YBR169C | 1.17 | 1.32 | 1.55 | 0.96 | 0.8 | 0.8 | 1.12 | 1.7 | 0.91 | 1.57 | 0.9 | 1.04 | 0.94 | 0.86 | 1.08 | 1.79 | 0.75 | 1.49 |
| YCL040W | 0.86 | 3.78 | 5.31 | 2.89 | 1.57 | 0.7 | 0.67 | 0.38 | 0.5 | 0.75 | 0.87 | 1.06 | 1.16 | 0.48 | 0.78 | 0.73 | 0.84 | 0.63 |
| YCR034W | 0.51 | 0.53 | 0.57 | 0.84 | 1.11 | 1.4 | 1.12 | 1.06 | 1.13 | 1.11 | 1.21 | 0.89 | 1.22 | 1.08 | 1.21 | 1.22 | 1.12 | 1 |
| YCR088W | 1.08 | 1.12 | 1.34 | 1.38 | 1.15 | 1.48 | 0.96 | 1.45 | 1.32 | 0.84 | 1.16 | 1.45 | 1.03 | 1.01 | 1.07 | 1.79 | 0.97 | 1.26 |
| YDL087C | 0.79 | 0.53 | 0.82 | 1.38 | 0.79 | 0.67 | 0.94 | 0.89 | 0.91 | 1 | 0.8 | 0.78 | 1 | 0.84 | 0.82 | 0.78 | 0.79 | 0.71 |
| YDL093W | 0.6 | 0.57 | 0.8 | 1.08 | 1.58 | 1.04 | 1.2 | 0.66 | 0.63 | 0.74 | 0.7 | 1.11 | 1.32 | 0.97 | 0.89 | 0.68 | 0.53 | 0.61 |
| YDL205C | 0.65 | 0.42 | 0.82 | 0.39 | 0.9 | 0.45 | 0.53 | 0.4 | 0.82 | 0.42 | 1.27 | 0.84 | 0.75 | 0.57 | 0.49 | 1.58 | 0.34 | 0.71 |
| YDR039C | 1.38 | 1.45 | 1.99 | 1.2 | 2.12 | 1.52 | 2.08 | 1.38 | 1.63 | 1.23 | 1.36 | 1.26 | 1.3 | 1.43 | 1.32 | 1.22 | 0.74 | 1.15 |
| YDR041W | 1.34 | 0.96 | 1.22 | 0.99 | 1.08 | 0.84 | 1.17 | 1 | 1.07 | 0.94 | 0.94 | 0.86 | 0.87 | 0.78 | 0.89 | 0.78 | 0.79 | 0.67 |
| YDR092W | 1.07 | 0.61 | 1.01 | 0.65 | 1.13 | 1.08 | 1.2 | 1.27 | 1.22 | 0.82 | 0.96 | 1.27 | 0.93 | 1.21 | 0.96 | 1.03 | 1.11 | 1.13 |
| YDR188W | 0.57 | 0.54 | 0.55 | 0.65 | 0.68 | 0.76 | 0.64 | 0.73 | 1.32 | 1.12 | 1.36 | 0.8 | 0.78 | 0.65 | 0.79 | 1.07 | 0.74 | 0.8 |
| YDR292C | 0.64 | 0.73 | 0.65 | 0.96 | 0.67 | 0.97 | 0.65 | 0.91 | 1.12 | 1.13 | 1.43 | 0.99 | 0.84 | 0.84 | 0.71 | 1.06 | 0.79 | 1.17 |
| YDR345C | 1.48 | 1.27 | 1.26 | 0.79 | 1 | 0.63 | 1.23 | 0.73 | 0.97 | 1.06 | 1.39 | 1.17 | 1.68 | 1 | 1.15 | 0.71 | 1.06 | 0.82 |
| YDR457W | 1.01 | 0.5 | 0.91 | 0.91 | 1.28 | 1.23 | 0.84 | 0.67 | 0.93 | 0.91 | 1.68 | 1.07 | 0.78 | 0.74 | 1.28 | 1.15 | 1.15 | 1.34 |
| YER008C | 0.57 | 0.75 | 0.86 | 0.7 | 0.93 | 0.79 | 0.97 | 0.89 | 0.99 | 0.78 | 0.78 | 1.2 | 0.87 | 0.86 | 1.07 | 0.99 | 0.91 | 0.89 |
| YER015W | 1.23 | 1.28 | 0.91 | 0.79 | 1.08 | 0.71 | 1.01 | 0.82 | 1 | 0.84 | 0.91 | 0.99 | 0.97 | 0.67 | 0.84 | 0.71 | 0.94 | 0.8 |
| YER091C | 0.73 | 2.08 | 1.3 | 0.6 | 0.38 | 1.86 | 2.01 | 2.18 | 1.36 | 0.84 | 0.96 | 0.84 | 0.64 | 0.61 | 0.94 | 1.77 | 0.89 | 1.04 |
| YER178W | 1.34 | 0.86 | 1.2 | 0.96 | 1.11 | 0.84 | 1.35 | 1.08 | 1.22 | 0.89 | 1.28 | 1.04 | 1.06 | 1.03 | 1.39 | 1.01 | 1.36 | 0.76 |
| YFL029C | 0.86 | 0.74 | 1.34 | 0.71 | 0.86 | 0.73 | 0.87 | 1.07 | 1.11 | 0.79 | 0.84 | 0.71 | 0.75 | 0.82 | 0.94 | 0.73 | 1.13 | 1.13 |
| YFR028C | 0.53 | 0.47 | 0.4 | 0.55 | 0.5 | 1.04 | 0.79 | 0.76 | 0.97 | 1.07 | 0.73 | 0.7 | 0.84 | 0.76 | 0.86 | 0.96 | 0.68 | 0.9 |
| YGL008C | 0.51 | 0.51 | 0.5 | 0.53 | 0.51 | 0.96 | 0.94 | 1.39 | 1.8 | 2.18 | 1.65 | 1.06 | 0.73 | 0.84 | 0.87 | 1.79 | 0.97 | 1.65 |
| YGL027C | 0.94 | 0.67 | 1.34 | 1.27 | 2.25 | 1.51 | 1.93 | 1.03 | 1 | 0.87 | 1.28 | 1.3 | 1.4 | 1.13 | 1.65 | 1.23 | 1.23 | 0.68 |
| YGL038C | 0.42 | 0.8 | 1.65 | 1.77 | 0.7 | 1.06 | 0.5 | 0.65 | 0.66 | 1.22 | 1.38 | 1.88 | 1.36 | 1.15 | 0.9 | 0.89 | 0.64 | 0.73 |
| YGL237C | 1.13 | 0.63 | 0.74 | 0.84 | 1.23 | 1.34 | 1.01 | 1.03 | 0.84 | 0.84 | 0.97 | 0.89 | 0.89 | 1.21 | 1.2 | 1.07 | 1.28 | 1.12 |
| YGR080W | 1.11 | 1.03 | 1.17 | 0.76 | 0.71 | 0.67 | 1.15 | 0.91 | 1 | 0.79 | 0.91 | 0.9 | 0.9 | 0.66 | 0.9 | 0.78 | 0.22 | 0.75 |
| YGR195W | 1.16 | 0.74 | 0.87 | 0.73 | 1.15 | 0.82 | 1.2 | 0.93 | 0.96 | 1.11 | 0.82 | 0.94 | 0.89 | 0.79 | 0.84 | 0.79 | 1.01 | 0.87 |
| YGR274C | 1.06 | 1 | 1.3 | 1.11 | 1.13 | 1.06 | 0.97 | 1.21 | 1.26 | 0.97 | 1.8 | 1.12 | 1.13 | 1.01 | 1.26 | 1.54 | 0.78 | 0.94 |
| YHL038C | 0.93 | 0.67 | 1.12 | 0.74 | 1.16 | 1.12 | 1.22 | 0.67 | 1.23 | 0.97 | 1.16 | 0.87 | 1.01 | 0.86 | 0.86 | 0.73 | 1.12 | 0.99 |
| YHR026W | 0.93 | 0.71 | 0.84 | 0.97 | 0.9 | 1.08 | 1 | 1.01 | 1.08 | 0.74 | 1.03 | 0.79 | 1.06 | 0.79 | 0.96 | 0.84 | 0.8 | 0.79 |
| YHR170W | 0.84 | 0.64 | 0.36 | 0.64 | 0.78 | 1.16 | 0.84 | 1.06 | 1.21 | 1.35 | 0.99 | 1 | 0.93 | 0.96 | 0.99 | 1.16 | 1.03 | 1.12 |
| YIL066C | 0.36 | 0.74 | 2.41 | 3 | 2.61 | 1 | 0.86 | 0.61 | 0.54 | 0.45 | 1.57 | 2.61 | 2.25 | 1.27 | 1.34 | 0.99 | 0.35 | 0.55 |
| YIL101C | 0.89 | 1.38 | 1.36 | 0.9 | 1.03 | 0.94 | 0.73 | 0.99 | 1.13 | 0.66 | 2.66 | 0.8 | 0.75 | 0.55 | 1.08 | 1.21 | 0.65 | 1 |
| YIR018W | 0.82 | 2.77 | 0.8 | 0.8 | 0.84 | 0.94 | 1.03 | 1.06 | 1.22 | 0.86 | 0.9 | 0.71 | 0.93 | 0.84 | 0.87 | 1.15 | 0.76 | 1 |
| YIR022W | 0.93 | 0.84 | 1 | 1.03 | 1.07 | 0.99 | 1.4 | 1.08 | 0.94 | 0.65 | 0.84 | 0.76 | 1.07 | 0.71 | 1.08 | 0.7 | 1.4 | 0.79 |
| YJL008C | 1.11 | 0.63 | 0.86 | 0.79 | 1.16 | 0.8 | 1.34 | 0.97 | 1.11 | 0.63 | 1.04 | 1 | 0.99 | 0.74 | 1.21 | 0.84 | 1.04 | 0.78 |
| YJL044C | 0.84 | 0.75 | 0.54 | 0.51 | 0.35 | 0.38 | 0.41 | 0.51 | 0.82 | 0.87 | 0.74 | 0.6 | 0.73 | 0.48 | 0.53 | 0.56 | 0.5 | 0.7 |
| YJL073W | 0.97 | 0.82 | 2.16 | 2.61 | 1.28 | 1 | 0.84 | 0.66 | 0.63 | 0.79 | 0.84 | 1.27 | 1.03 | 0.82 | 0.74 | 0.68 | 0.57 | 0.74 |
| YJL099W | 1.01 | 1.11 | 0.84 | 0.86 | 1.06 | 1.23 | 1.3 | 1.4 | 1.03 | 0.94 | 0.64 | 0.76 | 0.86 | 0.8 | 0.97 | 0.99 | 1.57 | 1 |
| YJL110C | 0.53 | 0.51 | 0.44 | 0.58 | 0.53 | 0.74 | 0.56 | 0.71 | 0.74 | 0.89 | 0.6 | 0.8 | 0.73 | 0.57 | 0.61 | 0.8 | 0.71 | 0.82 |
| YJL173C | 0.5 | 0.5 | 0.84 | 1.23 | 1.57 | 1.21 | 1.48 | 1.01 | 0.7 | 0.55 | 0.79 | 0.78 | 1.32 | 0.76 | 1.35 | 0.71 | 1.23 | 0.49 |
| YJL201W | 0.41 | 0.44 | 1.11 | 1.08 | 1.06 | 0.91 | 1.07 | 0.68 | 0.61 | 0.56 | 0.66 | 0.76 | 0.97 | 0.68 | 0.99 | 0.76 | 0.86 | 0.51 |
| YJR106W | 0.7 | 0.84 | 0.8 | 0.71 | 0.7 | 1.03 | 0.82 | 0.66 | 0.86 | 1.06 | 0.82 | 0.9 | 0.86 | 0.67 | 0.74 | 0.87 | 0.53 | 0.86 |
| YJR131W | 0.89 | 0.7 | 1 | 1 | 1.01 | 1.12 | 0.89 | 0.99 | 1.01 | 1 | 0.99 | 1 | 0.9 | 0.84 | 0.97 | 1.04 | 0.75 | 0.78 |
| YKL117W | 1.22 | 1.4 | 1.21 | 1.75 | 1.17 | 1.7 | 1.16 | 1.62 | 1.51 | 1.12 | 1.46 | 1.21 | 1.22 | 0.93 | 1.21 | 1.22 | 1.16 | 1.01 |
| YKL148C | 0.76 | 1.26 | 1.88 | 1 | 0.87 | 0.66 | 0.73 | 0.53 | 0.54 | 0.67 | 0.7 | 0.7 | 0.74 | 0.49 | 0.67 | 0.58 | 0.43 | 0.56 |
| YKL182W | 1.03 | 0.51 | 0.6 | 0.39 | 0.39 | 0.31 | 0.35 | 0.26 | 0.33 | 0.37 | 0.57 | 0.89 | 0.84 | 0.79 | 0.87 | 0.87 | 0.43 | 0.48 |
| YKL185W | 0.57 | 0.26 | 0.54 | 0.2 | 0.18 | 0.15 | 0.11 | 0.15 | 0.53 | 3.78 | 4.18 | 1.57 | 0.75 | 0.51 | 0.33 | 0.36 | 0.29 | 1.16 |
| YKR010C | 0.45 | 0.47 | 0.64 | 0.87 | 1.03 | 1.03 | 0.91 | 0.66 | 0.74 | 0.53 | 0.55 | 0.73 | 1.04 | 0.89 | 1 | 1.03 | 0.66 | 0.73 |
| YKR054C | 0.57 | 0.39 | 0.54 | 0.5 | 0.63 | 0.47 | 0.68 | 0.67 | 1.01 | 0.86 | 0.9 | 0.63 | 0.64 | 0.58 | 0.93 | 0.84 | 0.82 | 0.79 |
| YLR079W | 0.3 | 0.64 | 0.33 | 0.47 | 0.37 | 0.38 | 0.27 | 0.34 | 0.36 | 1.26 | 2.36 | 1.57 | 1.13 | 0.71 | 0.55 | 0.53 | 0.43 | 0.75 |
| YLR098C | 0.51 | 0.54 | 0.42 | 0.47 | 0.43 | 0.82 | 1 | 1.2 | 1.48 | 1.68 | 0.86 | 0.87 | 0.65 | 0.49 | 0.63 | 0.89 | 1 | 1.16 |
| YLR155C | 1.11 | 1.08 | 1.65 | 1.11 | 1.52 | 0.79 | 1.54 | 1.16 | 1.06 | 1.39 | 1.08 | 0.73 | 1.2 | 1.01 | 1.23 | 1.2 | 1.67 | 0.73 |
| YML035C | 0.96 | 0.66 | 1.36 | 1.12 | 1.35 | 0.94 | 1.32 | 0.93 | 1.32 | 1.15 | 1.23 | 0.91 | 0.96 | 0.67 | 1 | 0.82 | 1.13 | 0.82 |
| YML104C | 0.87 | 0.94 | 0.93 | 1.15 | 1.08 | 1.34 | 1.2 | 1 | 1.23 | 1.7 | 1.01 | 1.15 | 1.12 | 1.11 | 1.2 | 1.62 | 1.23 | 1.12 |
| YMR001C | 0.25 | 0.2 | 0.18 | 0.14 | 0.32 | 0.7 | 1.82 | 1.52 | 2.25 | 1.34 | 0.78 | 0.54 | 0.39 | 0.54 | 0.91 | 1.34 | 2.01 | 1.34 |
| YMR015C | 1.04 | 0.5 | 0.42 | 0.6 | 0.73 | 0.93 | 1.23 | 0.93 | 1.01 | 0.86 | 1.04 | 0.71 | 0.9 | 0.63 | 1.06 | 0.87 | 0.76 | 0.82 |
| YMR023C | 1.11 | 1.63 | 1.17 | 1.13 | 1.01 | 1.07 | 0.97 | 0.91 | 0.97 | 0.84 | 0.97 | 0.94 | 0.94 | 0.7 | 0.8 | 0.9 | 0.75 | 0.8 |
| YMR058W | 2.27 | 0.86 | 1.04 | 1.17 | 2.1 | 2.27 | 4.26 | 3.22 | 5.42 | 5.21 | 7.1 | 5.47 | 4.76 | 3.35 | 6.82 | 5.7 | 8.25 | 5.21 |
| YMR065W | 6.42 | 1.46 | 0.65 | 0.51 | 0.7 | 0.4 | 0.89 | 0.97 | 0.89 | 0.89 | 0.65 | 0.61 | 0.54 | 0.39 | 0.57 | 0.7 | 1 | 0.84 |
| YMR070W | 0.75 | 0.8 | 0.9 | 0.93 | 1 | 0.76 | 1.16 | 1.03 | 1 | 0.87 | 1.27 | 0.91 | 1 | 0.96 | 1.36 | 1.26 | 0.71 | 1.07 |
| YMR129W | 0.68 | 0.41 | 0.49 | 0.53 | 0.73 | 0.73 | 0.87 | 0.75 | 0.96 | 0.84 | 0.94 | 0.76 | 0.54 | 0.84 | 0.97 | 1.11 | 0.7 | 0.68 |
| YMR231W | 0.68 | 0.9 | 0.71 | 0.87 | 0.8 | 0.87 | 0.79 | 0.86 | 0.87 | 0.94 | 0.7 | 1.04 | 0.8 | 0.58 | 0.63 | 0.82 | 0.86 | 0.99 |
| YNL012W | 0.78 | 1.15 | 0.94 | 1.08 | 0.76 | 0.65 | 0.97 | 0.91 | 0.86 | 0.79 | 0.64 | 0.73 | 1.12 | 0.97 | 0.79 | 0.74 | 0.68 | 0.8 |
| YNL030W | 0.06 | 0.08 | 0.1 | 0.73 | 1.97 | 2.27 | 1.45 | 0.7 | 0.48 | 0.21 | 0.27 | 0.51 | 1.75 | 1.46 | 2.27 | 0.97 | 0.63 | 0.4 |
| YNL031C | 0.11 | 0.15 | 0.14 | 0.65 | 1.49 | 2.27 | 1.21 | 0.55 | 0.45 | 0.29 | 0.23 | 0.58 | 1.43 | 1.79 | 1.7 | 0.78 | 0.74 | 0.44 |
| YNL059C | 0.79 | 0.65 | 0.61 | 0.54 | 0.61 | 0.87 | 0.9 | 0.73 | 0.84 | 0.89 | 0.73 | 0.79 | 0.84 | 0.63 | 0.73 | 0.66 | 0.68 | 0.84 |
| YNL061W | 0.89 | 0.44 | 0.27 | 0.49 | 0.68 | 0.82 | 0.99 | 0.96 | 1.03 | 1.07 | 0.8 | 0.94 | 1 | 0.79 | 0.7 | 0.79 | 0.73 | 1.04 |
| YNL062C | 0.96 | 0.61 | 0.37 | 0.57 | 0.91 | 0.76 | 1.21 | 0.96 | 1.22 | 0.76 | 0.87 | 0.87 | 1.06 | 0.96 | 0.87 | 1.08 | 0.91 | 0.99 |
| YNL073W | 0.79 | 0.76 | 0.96 | 0.7 | 0.96 | 0.65 | 1.01 | 0.64 | 0.84 | 0.79 | 0.76 | 0.84 | 0.8 | 0.55 | 0.67 | 0.71 | 0.74 | 0.66 |
| YNL188W | 0.31 | 0.47 | 0.84 | 0.71 | 0.45 | 0.55 | 0.76 | 0.54 | 0.57 | 1.13 | 1.12 | 0.73 | 0.73 | 0.49 | 0.56 | 0.4 | 0.7 | 0.74 |
| YNL272C | 1.36 | 1.13 | 1.4 | 1.84 | 1.2 | 1.32 | 1.15 | 1 | 0.93 | 0.99 | 1.12 | 1.62 | 1.21 | 0.99 | 0.87 | 0.84 | 1.15 | 1.03 |
| YNR023W | 0.56 | 0.5 | 0.49 | 0.87 | 1.06 | 1.17 | 1.45 | 1 | 0.74 | 0.89 | 0.74 | 0.71 | 0.8 | 0.63 | 1.04 | 1.01 | 1.51 | 1.22 |
| YOL028C | 0.82 | 0.75 | 0.76 | 0.86 | 0.78 | 0.97 | 1.08 | 0.99 | 1 | 0.87 | 1.01 | 0.94 | 0.87 | 0.84 | 0.96 | 0.99 | 1.26 | 0.97 |
| YOL067C | 1.07 | 0.67 | 1.28 | 0.84 | 0.8 | 1.06 | 1.23 | 1.07 | 1.07 | 1 | 1.11 | 0.78 | 0.73 | 0.65 | 0.94 | 0.96 | 1.15 | 1.16 |
| YOL109W | 0.84 | 0.44 | 0.41 | 0.4 | 0.67 | 0.68 | 1.16 | 1.36 | 1.27 | 0.96 | 1.38 | 1.07 | 1.07 | 0.91 | 1.93 | 1.26 | 1.38 | 0.93 |
| YOR037W | 0.96 | 0.84 | 1.17 | 0.89 | 1.39 | 1.15 | 1.07 | 0.68 | 0.73 | 1.03 | 0.87 | 0.8 | 0.89 | 0.68 | 0.75 | 0.75 | 1.06 | 1.38 |
| YOR074C | 0.24 | 0.55 | 1.32 | 2.2 | 2.41 | 1.32 | 1.01 | 0.36 | 0.38 | 0.67 | 0.51 | 1.57 | 1.55 | 0.82 | 0.57 | 0.6 | 0.4 | 0.34 |
| YOR132W | 0.94 | 1.26 | 1.65 | 1.52 | 1.26 | 0.91 | 0.96 | 0.71 | 0.78 | 0.93 | 1 | 1.13 | 1.16 | 0.65 | 0.96 | 0.8 | 1.06 | 1.04 |
| YOR153W | 0.61 | 0.42 | 0.35 | 0.34 | 0.49 | 0.78 | 1.11 | 1.01 | 1.04 | 0.66 | 0.61 | 0.53 | 0.47 | 0.57 | 1.06 | 1.7 | 1.11 | 1.26 |
| YOR167C | 1.34 | 0.86 | 0.87 | 1.13 | 1.04 | 1.08 | 1.16 | 0.94 | 1.15 | 0.8 | 1.2 | 0.71 | 1.3 | 0.7 | 1.48 | 0.84 | 1.46 | 0.8 |
| YOR259C | 0.86 | 0.61 | 1.13 | 0.97 | 1.07 | 1.23 | 1.07 | 0.96 | 1.08 | 0.93 | 1.22 | 0.99 | 0.82 | 0.55 | 0.8 | 0.74 | 0.82 | 0.8 |
| YOR261C | 0.9 | 0.57 | 0.9 | 1 | 0.96 | 1.23 | 0.87 | 0.78 | 1.03 | 0.86 | 1.21 | 0.76 | 0.76 | 0.49 | 0.76 | 0.6 | 0.9 | 0.65 |
| YOR321W | 0.61 | 0.66 | 1.06 | 2.1 | 1.57 | 1.34 | 1.32 | 0.76 | 0.66 | 0.54 | 0.8 | 1.17 | 1.4 | 0.96 | 1.04 | 0.87 | 0.79 | 0.54 |
| YPL040C | 0.68 | 0.75 | 0.79 | 1.12 | 0.94 | 0.75 | 0.9 | 0.71 | 0.9 | 0.99 | 0.9 | 0.99 | 1.01 | 0.64 | 0.61 | 0.84 | 0.61 | 0.79 |
| YPL050C | 0.86 | 0.64 | 1.16 | 1.11 | 1.34 | 1.07 | 1.36 | 1.07 | 1 | 0.86 | 0.86 | 0.84 | 1.07 | 0.87 | 1.01 | 0.75 | 0.94 | 1.04 |
| YPL061W | 1 | 2.66 | 5.42 | 2.89 | 1.46 | 0.91 | 0.87 | 1.04 | 1.23 | 1.4 | 1.97 | 1.11 | 0.63 | 0.34 | 0.35 | 0.43 | 0.64 | 0.71 |
| YPL072W | 0.93 | 0.99 | 1.06 | 1.17 | 1.04 | 1.68 | 1.52 | 1.48 | 1.01 | 0.86 | 0.66 | 0.87 | 1.01 | 0.78 | 1.11 | 0.96 | 1.43 | 1.48 |
| YPL086C | 0.91 | 0.48 | 0.37 | 0.64 | 0.76 | 1.04 | 1.22 | 1.17 | 1.13 | 0.9 | 0.66 | 0.82 | 0.8 | 0.82 | 0.64 | 0.68 | 0.84 | 0.86 |
| YPL092W | 1.35 | 4.39 | 2.18 | 1.28 | 1 | 0.61 | 0.66 | 0.66 | 0.79 | 0.75 | 0.7 | 0.54 | 0.6 | 0.54 | 1 | 0.68 | 0.51 | 0.67 |
| YPL127C | 0.12 | 0.14 | 0.64 | 1.54 | 2.18 | 2.36 | 2.05 | 1.21 | 0.74 | 0.47 | 0.41 | 0.91 | 1.38 | 1.57 | 1.34 | 1.38 | 1.17 | 0.73 |
| YPL234C | 0.78 | 0.58 | 0.44 | 0.7 | 0.7 | 0.57 | 0.94 | 0.64 | 0.76 | 0.41 | 0.6 | 0.45 | 0.71 | 0.45 | 0.84 | 0.41 | 0.53 | 0.44 |
| YPR056W | 0.6 | 0.51 | 0.68 | 0.54 | 0.86 | 0.84 | 0.89 | 0.68 | 0.73 | 0.78 | 0.86 | 0.67 | 0.79 | 0.65 | 0.76 | 0.76 | 0.99 | 0.9 |
| YPR102C | 1.15 | 0.84 | 1.03 | 1.08 | 1.06 | 1.16 | 1.13 | 1.23 | 1.51 | 0.99 | 1.51 | 0.89 | 1.12 | 0.76 | 1.7 | 1.13 | 1.9 | 1.08 |

### Example 4

The data set for this example is listed in Table 2 and is an extract of the data set described in Alizadeh, A. A., et al. (2000) Distinct types of diffuse large B-cell lymphoma identified by gene expression profiling. Nature 403:503-511.

The data set generated from the microarray experiments described in the above paper can be obtained from the following web site:

http://genome-www4.stnford.edu/MicroArray/SMD/publications.html

There are n=100 genes and n=42 samples. In the following DLBCL refers to “Diffuse large B cell Lymphoma”. The samples have been classified into two disease types GC B-like DLBCL (21 samples) and Activated B-like DLBCL (21 samples). The design matrix T has 1 column with values −1 if the sample is in group 2 and +1 if the sample is in group 1. This array data is used to illustrate the potential use of the method of the present invention in discovering genes which are diagnostic of different disease types.

The results of applying the above methodology are given below along with a (partial) list of potentially diagnostic genes. The plot shows factor loadings calculated for each array, with a Box plot showing the distribution of factor loadings from each disease type. Note the distinct factor loadings for each grouping in the plot.

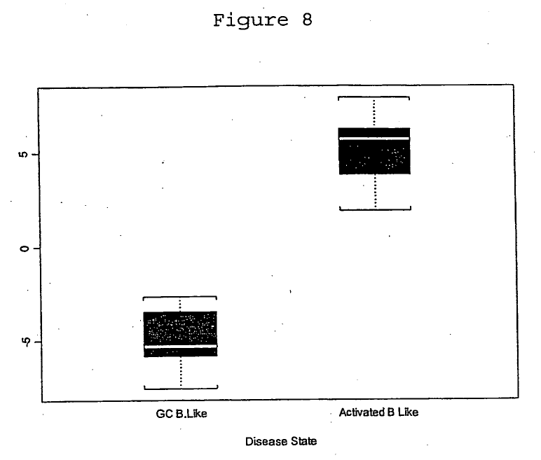

The genes identified are shown below. Results of the gene expression from these genes is shown in FIG. 8.

Canonical Variate1
d = 0.912 p-value = 0.000

| Gene | Score | p-Value |
|---|---|---|
| GENE2238X | 0.4491 | 0.027 |
| GENE2943X | 0.4102 | 0.045 |
| GENE2977X | 0.3827 | 0.024 |
| GENE1246X | 0.4157 | 0.030 |
| GENE124X | 0.4213 | 0.012 |
| GENE122X | 0.3318 | 0.038 |
| GENE1614X | −0.4406 | 0.038 |

TABLE 2

| RowNames | DLCL0001 | DLCL0002 | DLCL0003 | DLCL0004 | DLCL0005 | DLCL0006 | DLCL0007 | DLCL0008 | DLCL0009 | DLCL0010 | DLCL0011 | DLCL0012 | DLCL0013 | DLCL0014 |
|---|---|---|---|---|---|---|---|---|---|---|---|---|---|---|
| GENE3950X | −0.2049 | 0.6574 | −0.3501 | 1.1837 | 0.3306 | 0.1310 | 1.5559 | −0.4136 | 0.8026 | 0.0583 | −0.0415 | −1.3484 | 0.6846 | −0.7494 |
| GENE2531X | −0.2116 | 1.0063 | −0.4699 | 1.1355 | 0.5358 | 0.0929 | 1.2739 | −0.5714 | 0.3974 | −0.0178 | 0.2498 | −1.6693 | 0.6096 | −1.1711 |
| GENE918X | −0.1815 | 0.9708 | −0.3538 | 1.1432 | 0.3901 | 0.4990 | 1.2520 | −0.6532 | 1.0615 | 0.2813 | −0.1996 | −1.6149 | 0.7077 | −0.9254 |
| GENE3511X | −1.2609 | −0.3673 | 0.2774 | 0.6506 | 0.2095 | −0.6501 | −0.0393 | −1.9622 | −0.3786 | −1.3288 | −0.0167 | 0.3113 | 0.9334 | 0.2435 |
| GENE3496X | −1.5438 | 0.2235 | 0.3742 | 0.6152 | 0.0026 | 0.4043 | 0.7658 | −2.1362 | 0.2235 | 0.0930 | 0.1131 | −0.0175 | 0.6352 | 0.8963 |
| GENE3484X | −1.5441 | 0.2644 | 0.3324 | 0.5755 | 0.3227 | 0.3810 | 0.6922 | −2.0400 | 0.5074 | −0.0857 | 0.3713 | −0.2315 | 0.5852 | 0.6241 |
| GENE3789X | −0.8190 | 0.8721 | −0.4551 | −0.3695 | 0.5510 | 0.8935 | −0.5408 | −1.8466 | 0.5510 | 0.3155 | 0.6152 | −0.5194 | 1.7283 | −0.9261 |
| GENE3692X | 1.5834 | −1.3890 | 0.2694 | 0.3204 | −0.9297 | −0.8659 | −0.0240 | 1.2389 | −0.3046 | 1.0093 | −0.3812 | −0.0623 | −2.2564 | −0.0240 |
| GENE3752X | −0.5429 | 0.0079 | 1.0622 | 1.0307 | 0.4799 | 0.3226 | −0.0708 | −1.5657 | −0.0393 | −1.8490 | −0.2439 | −0.9048 | 0.4957 | 1.1094 |
| GENE3740X | −0.1202 | 0.3514 | −0.2352 | 0.5584 | −0.7183 | 1.7546 | 1.1220 | −2.1561 | −0.2697 | −1.1094 | 0.0178 | −0.1547 | −0.9484 | −0.6953 |
| GENE3736X | −1.0454 | 0.1940 | 0.1413 | 1.0247 | 0.4182 | 1.0642 | 0.0622 | −2.0475 | −0.0697 | −1.2827 | 0.1940 | −0.4389 | −0.2411 | −0.4125 |
| GENE3682X | 0.0352 | −0.5229 | −1.0198 | −1.0882 | −0.7605 | 1.2054 | 0.8310 | −1.0306 | −0.4040 | −0.5625 | −1.1098 | 0.7770 | 2.0876 | −0.2384 |
| GENE3674X | 0.0919 | −0.3555 | −1.1076 | −0.8632 | −1.0361 | 0.9907 | 1.1110 | −0.8782 | −0.1675 | −0.6977 | −0.5699 | 0.6898 | 2.2127 | −0.0660 |
| GENE3673X | 0.4663 | −0.7188 | −1.0865 | −1.3763 | −0.7102 | 0.9291 | 0.8167 | −1.3677 | −0.3598 | −0.7707 | −0.9265 | 1.0286 | 0.3668 | 0.0511 |
| GENE3644X | 1.2679 | 1.0367 | −0.2156 | 0.4202 | 0.5551 | −0.1771 | 0.5743 | −1.2367 | −0.2349 | −1.4101 | 0.5551 | −1.4872 | 0.8248 | −1.5257 |
| GENE3472X | −0.5140 | 0.4945 | 0.5546 | 0.2904 | −0.0097 | 1.2149 | 1.1549 | −2.0388 | −0.6340 | −0.9102 | 0.8667 | −0.6941 | 1.1189 | −1.1503 |
| GENE2530X | −0.3729 | −0.7347 | −0.5176 | −0.0474 | 0.2601 | 0.0612 | −0.2102 | −1.2411 | −0.2825 | −1.4401 | −0.4091 | −0.0474 | −0.2463 | 0.4048 |
| GENE2287X | −0.7046 | −0.7689 | −0.4475 | 0.4799 | −0.3006 | 0.6084 | 0.8196 | −1.2739 | 0.2228 | −1.0995 | −0.0894 | 0.5442 | −0.4567 | −0.3098 |
| GENE2328X | −0.4273 | 0.4495 | −1.8079 | −1.0243 | 0.4682 | 0.7853 | −2.0504 | −0.9683 | −0.0915 | 0.2816 | 0.2443 | −0.4646 | 2.0913 | 0.3562 |
| GENE2417X | −1.1810 | 1.0531 | 0.1474 | 0.1021 | 0.4644 | 2.0191 | 0.7210 | −1.1055 | −0.9546 | −2.2226 | 2.1701 | 0.6757 | 1.6418 | −0.0791 |
| GENE2238X | 0.6934 | −0.2178 | 0.8979 | 0.6190 | −0.3294 | 0.2843 | −0.3294 | −0.0319 | 0.8979 | −0.2550 | 0.8794 | 0.5818 | −0.5898 | −1.9287 |
| GENE1971X | −0.1957 | 1.3122 | −0.3276 | −0.2145 | 1.4441 | 0.3132 | 0.8221 | −0.9873 | 0.0494 | −1.0815 | 0.0117 | −0.8365 | 1.1048 | −0.6480 |
| GENE3086X | 0.0236 | −1.4920 | −0.3702 | 0.2026 | −0.0600 | −0.7521 | −0.6089 | −0.1674 | 0.7873 | 1.5034 | −0.6686 | −0.4776 | −0.7760 | −0.1793 |
| GENE1009X | 1.4548 | −0.6280 | 0.7398 | 0.2580 | 0.1025 | −0.3483 | −0.5970 | −0.3793 | −0.5659 | 1.1750 | −1.1876 | 0.8642 | −0.9389 | −0.0063 |
| GENE1947X | 0.4856 | −0.5274 | 0.1845 | 0.1023 | −0.5000 | −0.1441 | 1.4713 | 0.9237 | 0.7321 | 0.8689 | −0.1714 | 2.2105 | 0.1023 | −1.3214 |
| GENE3190X | 2.0024 | −0.8814 | 0.8489 | −0.6571 | −0.3047 | −0.2299 | −1.0417 | 1.4577 | 0.0585 | 1.5218 | −0.3794 | 0.1760 | −0.4969 | −0.0270 |
| GENE3379X | 0.7059 | −0.4788 | 1.6020 | 0.0224 | −0.3117 | 0.2351 | −0.6762 | 1.2223 | 0.6451 | 0.9489 | 0.2806 | 0.0832 | 0.9793 | −0.9496 |
| GENE3184X | 1.3782 | −0.6784 | 0.9336 | 0.8335 | −0.5783 | −0.7117 | −0.1337 | 0.7334 | 0.3777 | −1.3232 | −0.6784 | 2.7901 | −0.2782 | −0.1448 |
| GENE3122X | 1.1454 | −0.5556 | −0.3894 | 1.2236 | −0.4089 | −0.4676 | 0.9890 | 0.6175 | 0.9694 | 0.8619 | 0.2949 | 0.9205 | −0.3894 | −1.6700 |
| GENE1099X | 0.5601 | −0.8521 | −0.7039 | 0.5133 | −0.5634 | −1.0082 | −0.8521 | 1.3871 | 0.6927 | 0.7786 | 0.0139 | −0.4620 | 0.6771 | 0.0607 |
| GENE3032X | 0.5833 | −1.4015 | −0.4815 | 0.6600 | −0.4134 | −0.9415 | −0.9245 | 1.4352 | 0.7111 | 0.7793 | 0.0381 | −0.7030 | −0.1152 | 0.1830 |
| GENE2675X | 0.3661 | −1.0045 | 0.6262 | 1.8668 | −0.7244 | −1.1245 | −0.3842 | 2.1269 | −0.5743 | 2.0568 | −0.4642 | −0.3742 | 0.2361 | −0.5843 |
| GENE2481X | 0.4123 | −0.8389 | 0.7840 | 1.8267 | −0.5487 | −1.0111 | −0.3130 | 2.0443 | −0.1498 | 2.1078 | −0.4943 | −0.2949 | 0.3398 | −0.9930 |
| GENE2878X | 1.0922 | −0.8274 | 0.2785 | 0.9566 | 0.3202 | −0.5875 | −1.2238 | 1.3530 | 1.3008 | 0.2367 | −0.6188 | 0.0594 | −0.4727 | −0.9735 |
| GENE2943X | 1.5951 | −0.6212 | 0.3013 | 1.0551 | 0.7063 | −0.5649 | −1.1162 | 1.6288 | 1.3026 | 0.2226 | −0.6774 | 0.8188 | −0.9474 | −0.4637 |
| GENE2977X | 1.2805 | −1.2491 | 1.1314 | 1.1262 | −0.6527 | −1.1000 | −0.8275 | 0.9463 | −0.1129 | 0.1905 | −0.7298 | 0.6584 | −1.4702 | −0.5756 |
| GENE3014X | 1.9501 | −1.2171 | 0.4584 | 0.7935 | −0.2875 | 0.0476 | −1.2603 | 2.0582 | 0.5665 | −1.4441 | −0.8712 | −0.8083 | −0.0064 | −0.1037 |
| GENE2006X | 0.3456 | −1.0625 | 0.2272 | 1.4378 | −0.1939 | −0.6677 | −0.6414 | −0.6545 | 0.0298 | 2.6616 | −0.7335 | 0.5561 | −0.3782 | 0.0298 |
| GENE1368X | 0.5254 | −0.4359 | 1.7741 | 1.1000 | −0.2591 | −1.3642 | 0.3928 | 0.7243 | 0.2271 | 1.4978 | 0.2271 | 0.7906 | −0.7564 | −0.6127 |
| GENE1184X | 0.5950 | −0.5359 | 1.7039 | −0.8914 | −0.0308 | −1.3154 | 0.4962 | 0.7487 | 0.2107 | 1.3306 | 0.1778 | 0.7267 | −0.7225 | −0.5249 |
| GENE1226X | 1.1537 | −1.1220 | −0.3129 | −0.0769 | −0.5994 | −0.2454 | −0.8944 | 1.6342 | 0.9514 | 0.6480 | 0.5131 | 1.3054 | −1.8132 | −0.2370 |
| GENE1228X | 1.1347 | −0.3684 | 1.9013 | −0.9074 | 0.7934 | −0.1948 | 0.1286 | −0.6140 | −0.8176 | 2.3265 | 0.9072 | 0.5718 | 0.2184 | 0.0268 |
| GENE1231X | 0.2407 | −1.2858 | 0.0103 | 1.6088 | −0.8538 | 0.2551 | −0.3785 | 0.5575 | 0.5575 | 0.0823 | 1.3640 | −0.0761 | −0.8970 | −1.4730 |
| GENE1246X | 0.3136 | −1.0667 | 0.3136 | 1.6182 | −0.6627 | 0.4567 | −0.7553 | 0.9449 | 0.3136 | −0.1998 | 0.2968 | 0.1285 | −1.4118 | −2.0767 |
| GENE1172X | 0.0021 | −0.6792 | 0.5580 | 1.1317 | 0.0918 | 0.4862 | −1.3336 | 0.5938 | −0.0875 | 0.5221 | −0.3923 | 0.6566 | −2.1136 | −2.9653 |
| GENE1164X | −0.3385 | −0.6039 | −0.3053 | 1.0383 | 0.6568 | 0.1923 | −2.0636 | 0.3914 | 0.1758 | 0.7729 | −0.3551 | 0.2587 | −1.6323 | −0.6371 |
| GENE3029X | 0.9558 | −1.8240 | −0.4890 | −0.0318 | −0.2512 | 0.4803 | −0.1415 | 0.6997 | 0.6997 | 1.4861 | 0.2060 | 0.5900 | 0.9740 | 0.3705 |
| GENE1027X | 0.3195 | −0.8192 | −0.0407 | 1.1561 | −0.7030 | 1.1329 | −0.1220 | 1.5396 | −0.0639 | 0.8656 | 0.0871 | 1.3304 | −1.0748 | 1.2026 |
| GENE1354X | 1.0921 | 0.3968 | 0.5090 | 0.4192 | −0.3883 | −0.0967 | −0.7247 | 0.4641 | −0.0742 | 0.0379 | −0.3883 | 0.0603 | −0.4780 | 0.7108 |
| GENE62X | −1.7087 | −0.3336 | −0.2409 | 0.6397 | 0.5470 | −0.1173 | 0.0063 | 2.1229 | 0.8869 | −1.0752 | −0.1019 | 0.6551 | −0.4572 | −1.0752 |
| GENE932X | −1.6636 | 0.1194 | −0.3264 | −1.7472 | −0.6050 | −0.4935 | −0.1592 | −1.4407 | −1.0786 | −0.7721 | −0.1035 | 0.3701 | −0.0199 | 0.2587 |
| GENE3611X | −1.3618 | 0.5350 | −0.5350 | 0.3161 | −0.1702 | −0.7052 | 1.4590 | −1.3131 | −0.5836 | −2.9911 | 0.5107 | −1.4834 | 0.7052 | 0.6566 |
| GENE3631X | −0.5379 | 0.4721 | −0.9278 | 0.0823 | 0.0291 | 1.3404 | −0.0418 | −1.7783 | −0.2898 | −0.8923 | 0.3126 | −1.3708 | −0.0772 | 0.1354 |
| GENE330X | 0.8497 | 0.6081 | −1.5880 | −0.7095 | −0.9511 | 1.1132 | 0.5422 | −0.9731 | 0.7179 | −1.2366 | 1.2669 | −2.6860 | −0.0946 | −1.1048 |
| GENE331X | −0.8855 | 0.8435 | −0.4014 | −0.4878 | −0.0037 | 1.0510 | 0.1519 | −1.3870 | 0.6706 | −1.3524 | 1.5179 | −1.7155 | 2.8839 | −0.5570 |
| GENE808X | 1.5424 | −0.0178 | −0.2335 | 0.7125 | 0.4137 | 0.4469 | −0.1672 | −0.5157 | 1.0278 | 1.0444 | 1.2104 | −0.2833 | −0.4659 | −0.8145 |
| GENE487X | 1.1631 | −0.5281 | 0.2915 | 0.0053 | 1.2932 | −0.5802 | −0.3330 | 0.3565 | −0.1378 | 1.1761 | −1.1786 | 1.4493 | −0.5281 | −0.8664 |
| GENE621X | 0.8961 | −0.7734 | 0.2879 | −0.0341 | 1.1465 | −0.1772 | −0.6422 | 0.3117 | −0.4395 | 1.4088 | −0.9403 | 1.3611 | −0.8330 | −0.5468 |
| GENE622X | 1.2278 | −0.3796 | 0.3532 | 0.2113 | 0.6132 | −0.4269 | 0.2350 | −0.6751 | −0.1669 | 1.6533 | −1.1360 | 1.1923 | −0.8051 | −0.8642 |
| GENE634X | −1.6102 | 0.9498 | −0.4669 | 0.6888 | 0.7261 | 0.1296 | 0.8877 | −2.0328 | 0.2663 | 0.5770 | 0.5024 | −0.6782 | 0.1793 | 0.0675 |
| GENE659X | −1.0282 | 2.0564 | −0.1360 | 0.7435 | 0.1317 | 0.1062 | 1.2916 | −1.7165 | −0.2634 | −1.3723 | 1.8652 | −0.5821 | 1.4828 | 1.0877 |
| GENE669X | −0.7541 | 1.9543 | −0.0171 | 0.8396 | 0.2500 | 0.1487 | 1.4108 | −1.9056 | −0.0724 | −1.0673 | 1.7701 | −1.0120 | 1.4016 | 1.0147 |
| GENE674X | −0.7844 | 2.0333 | 0.2374 | 0.7844 | 0.6606 | 0.1858 | 0.8567 | −1.9094 | −0.3716 | −1.5379 | 1.4656 | −0.8360 | 1.4553 | 1.1663 |
| GENE675X | −1.8669 | −0.3961 | 0.5014 | 0.2751 | −0.2528 | 0.2676 | 1.0520 | −2.2591 | −0.4037 | −0.5998 | 0.0790 | −0.3358 | 0.9539 | 1.0972 |
| GENE676X | 0.1521 | 2.9355 | −0.8281 | −0.0536 | 0.0553 | 3.1896 | −0.4045 | −0.6466 | −0.7192 | −0.7676 | 0.1642 | −0.0899 | 0.4063 | −0.1262 |
| GENE704X | −0.2724 | 0.8058 | −0.6828 | −0.4656 | 0.0977 | 0.0253 | −1.2139 | −1.2219 | 0.1782 | 0.0575 | −0.4977 | −0.9484 | 0.0253 | −0.4253 |
| GENE734X | −0.1106 | 0.8918 | −0.7138 | −0.3740 | −0.0512 | 0.0593 | −1.0536 | −1.4104 | 0.3566 | −0.3485 | −0.2551 | −1.3254 | −0.0087 | −0.3060 |
| GENE738X | −0.3670 | 1.1934 | −0.4616 | −0.9817 | 2.0445 | 1.2643 | −0.2488 | −2.2347 | 0.7914 | −1.1472 | 1.1461 | −0.2488 | 0.4605 | −1.3127 |
| GENE456X | 0.2548 | 1.4336 | 0.2701 | −0.8322 | 0.1017 | 0.1936 | −1.5211 | −1.4752 | 0.2395 | −1.3068 | 0.3007 | −0.7097 | 1.1274 | 0.2701 |
| GENE744X | −0.1761 | 1.0752 | 0.2892 | −1.2991 | 0.9309 | −0.1440 | −1.1066 | −1.5237 | −0.3526 | −0.9622 | 0.1448 | −0.7536 | 1.3801 | 0.4014 |
| GENE179X | −1.5071 | −0.2186 | −3.7390 | −0.3566 | −0.8398 | 0.7018 | 0.2416 | −0.7248 | −0.5177 | −1.4381 | 0.2186 | −0.0575 | 0.0805 | −0.9319 |
| GENE124X | −1.3867 | 1.3179 | −0.7428 | −0.7714 | −0.5997 | 0.5595 | −0.1704 | −2.4027 | −0.1560 | −0.8000 | 0.2446 | −0.3135 | 1.4753 | −0.1274 |
| GENE122X | −1.2443 | 1.2153 | −0.7888 | −0.4396 | −0.7736 | 0.4410 | −0.1815 | −2.6107 | −0.0296 | −1.1076 | 0.4410 | −0.8799 | 1.3975 | 0.3044 |
| GENE111X | −0.7042 | 0.8689 | −1.0433 | −0.3245 | −1.0840 | 0.6790 | 0.7469 | −2.1418 | −0.0262 | −0.9483 | 0.6112 | −0.7449 | 1.5606 | 0.4892 |
| GENE97X | −0.1985 | 1.1612 | 0.2602 | −0.4770 | −0.5589 | 0.0472 | 0.5223 | −1.8532 | −0.1822 | −1.7549 | −0.6409 | −1.1651 | 0.3912 | 0.3912 |
| GENE2645X | −1.0298 | 1.1902 | 0.0604 | −0.3955 | 0.6749 | −0.0585 | −0.7324 | −1.5055 | 0.7145 | −1.6046 | 0.5163 | −0.2567 | 1.2893 | 1.1704 |
| GENE3408X | 0.6893 | −0.4665 | 0.5792 | −0.5766 | −0.3748 | 0.2306 | −1.0719 | −0.7600 | −0.2830 | 1.9551 | −0.0079 | 0.2123 | −1.2187 | −1.6589 |
| GENE3854X | 0.6938 | −0.9260 | 0.4181 | −0.2884 | −0.2884 | 0.3492 | −0.8399 | −0.6331 | −0.5814 | 1.8312 | 0.0734 | 0.6421 | −1.1845 | −2.1668 |
| GENE1406X | 0.0021 | −0.9105 | 0.4473 | −0.3540 | −0.1314 | 0.6254 | −1.7563 | −0.0647 | 0.3805 | 0.0689 | −0.9105 | 0.7589 | −1.0886 | −0.1760 |
| GENE1401X | 1.7535 | −0.9049 | 0.7783 | 1.4704 | −0.8419 | −0.1655 | 0.2749 | 2.0839 | −0.5903 | 0.0861 | −1.1251 | 1.1558 | −0.8419 | −1.2824 |
| GENE3462X | −0.3011 | 0.2070 | 0.1129 | −0.3952 | −0.6774 | −1.0914 | 1.2231 | −0.0376 | −0.5269 | −1.1478 | −0.9785 | −1.1102 | 1.0726 | 0.3199 |
| GENE3173X | −0.5215 | −0.2846 | 0.3418 | −0.2168 | −0.0476 | −0.4369 | 0.9681 | −1.3849 | −1.9774 | −0.7247 | −0.4200 | 0.7311 | 0.1217 | 0.3249 |
| GENE3971X | 1.5198 | −0.5224 | −0.2014 | 0.6154 | −1.5434 | 0.1486 | −0.4640 | −0.2306 | 0.7613 | 1.3156 | 0.7321 | 0.0903 | −0.2598 | −0.8724 |
| GENE1756X | 1.0949 | −1.9916 | 1.4067 | −0.1054 | −1.3369 | −0.7134 | 1.0326 | 0.5181 | −1.1498 | 1.4846 | −1.0563 | 0.1908 | −1.2122 | −0.8225 |
| GENE1533X | 1.5099 | −1.6932 | 1.1189 | 0.3219 | −1.7534 | −0.4601 | 0.6527 | 0.7430 | −0.2646 | 1.4949 | −0.6105 | 0.0963 | −0.9263 | −1.0315 |
| GENE1757X | 0.6631 | −0.7090 | 0.0789 | 0.0382 | −0.6275 | −0.2607 | 0.0518 | 1.4647 | 0.1061 | 1.8722 | −0.3286 | 1.1658 | −1.4019 | −0.6547 |
| GENE3572X | 0.5991 | −0.5067 | 1.0958 | 0.6151 | 0.3106 | −1.5484 | −0.6509 | 0.6952 | −0.2663 | 1.8330 | −0.0420 | 0.1984 | −1.2279 | 0.1984 |
| GENE3571X | −0.5755 | −0.4997 | 0.6209 | −0.8935 | 0.7269 | −0.0303 | −0.4392 | −1.4841 | −0.9238 | −1.3932 | 0.0454 | 0.2120 | 1.4841 | −0.1817 |
| GENE385X | −1.2426 | 0.7899 | −0.2381 | −0.2614 | −0.7287 | 0.9300 | 0.3693 | −2.0603 | −0.7754 | 0.0656 | −0.1446 | 0.5095 | 0.9768 | 0.4394 |
| GENE1614X | −1.7405 | 1.2328 | 0.2134 | −0.9335 | −0.0627 | 1.0204 | −0.2114 | −1.6131 | −1.0821 | 0.0647 | −0.2963 | 1.0204 | 0.7656 | 0.1922 |
| GENE1623X | −0.9216 | 0.5149 | 0.6527 | −1.4136 | 1.2233 | 0.0623 | 0.2197 | −0.1935 | −0.0164 | −0.4100 | 0.2788 | 1.1053 | 1.0462 | 0.3378 |
| GENE1646X | −1.0213 | 0.3776 | −0.5812 | −0.7383 | −0.0939 | 0.6291 | −0.8641 | −1.1941 | −0.1882 | −1.1784 | 0.4090 | 0.0161 | 2.2794 | 0.1890 |
| GENE1660X | 0.9611 | −0.4493 | −0.6750 | 0.3687 | −0.9711 | −0.6891 | −0.1672 | 0.8200 | −0.2236 | 1.8073 | −0.9288 | 0.7072 | −0.9994 | −0.5480 |
| GENE1721X | 0.9852 | −0.1574 | −0.3398 | 0.4503 | −1.3366 | −0.2668 | −0.2547 | 0.1586 | 0.0249 | 1.5808 | −1.3001 | 0.6327 | −0.6923 | −0.8260 |
| GENE1573X | −0.0220 | 0.9123 | −0.0901 | −0.1485 | 0.1434 | 0.7079 | 0.4646 | −1.4721 | −0.8298 | 0.7371 | −0.6351 | −1.0244 | 0.8539 | −0.5475 |
| GENE1553X | −0.7350 | 2.0362 | 0.5313 | −0.4230 | −0.2211 | 0.9167 | −0.3863 | −1.1938 | −1.5425 | 0.1643 | −0.0192 | 1.3572 | 1.1003 | −0.2211 |
| GENE1773X | −1.1428 | 2.1206 | 0.1544 | −0.7780 | −0.3726 | 0.7625 | −0.7982 | −1.6698 | −0.9401 | 0.3774 | 0.4382 | 0.7220 | 0.7220 | −0.6563 |
| GENE913X | 1.0593 | 1.2244 | 1.0593 | 0.4492 | 0.2195 | −1.2880 | −0.7568 | −0.4768 | 0.4635 | 0.3056 | 0.6717 | 0.5353 | −1.1588 | −0.5414 |
| GENE3980X | 0.9547 | 1.3890 | 1.1508 | 0.3454 | 0.2613 | −1.1745 | −0.9644 | −0.3480 | 0.1913 | 0.3664 | 0.3314 | 0.7166 | −1.2586 | −1.2360 |
| GENE3X | −0.0042 | 2.4527 | −0.8465 | 0.0485 | 0.6276 | 0.9786 | −0.0744 | −2.2329 | −0.3727 | 1.1541 | −0.1972 | −0.7237 | 0.6802 | 0.2415 |
| RowNames | DLCL0015 | DLCL0016 | DLCL0017 | DLCL0018 | DLCL0020 | DLCL0021 | DLCL0023 | DLCL0024 | DLCL0025 | DLCL0026 | DLCL0027 | DLCL0028 | DLCL0029 | DLCL0030 |
| GENE3950X | −0.1686 | 0.1582 | 0.8207 | −0.0959 | 0.5847 | 0.3942 | −1.0761 | −0.3501 | 0.7300 | −1.5572 | 0.1491 | 0.5847 | 0.2126 | 0.7753 |
| GENE2531X | −0.4330 | 0.0837 | 1.1909 | −0.0732 | 0.4712 | 0.2313 | −1.2726 | −0.3869 | 0.7849 | −1.3741 | 0.1944 | 0.4897 | 0.2313 | 0.8772 |
| GENE918X | −0.3448 | 0.1452 | 1.2248 | −0.1633 | 0.5534 | 0.4173 | −1.4063 | −0.3266 | 0.7712 | −1.1795 | 0.1996 | 0.6442 | 0.0998 | 0.6351 |
| GENE3511X | −0.6162 | −0.5370 | 2.2002 | −0.7180 | −0.8876 | 1.8270 | 0.5602 | 0.3453 | 0.9221 | −0.6840 | 1.1257 | 1.1483 | −0.1185 | 0.1530 |
| GENE3496X | −1.6743 | 0.4645 | 2.5230 | −1.4735 | 0.4645 | −0.3689 | 0.0930 | −0.1480 | 1.4486 | −0.7003 | 0.4043 | 0.6252 | 0.1030 | −0.2183 |
| GENE3484X | −1.6802 | 0.3130 | 2.3548 | −1.5149 | 0.3227 | −0.4454 | −0.1148 | −0.4065 | 1.2464 | −0.7468 | 0.2060 | 0.8575 | 0.1963 | −0.0079 |
| GENE3789X | −1.3542 | 1.0861 | 2.9271 | −0.6264 | 0.4439 | 1.1289 | −0.8405 | −0.4551 | 0.3583 | 0.2727 | 0.3583 | 0.8721 | −0.6264 | 0.4439 |
| GENE3692X | 1.8385 | −1.6824 | −1.2869 | 1.1879 | 0.3970 | 1.2517 | −0.6873 | 0.0015 | 0.4225 | 0.7159 | −1.0318 | −0.1771 | −0.3939 | −0.0495 |
| GENE3752X | −1.7073 | −0.9363 | 3.1393 | −0.1967 | 0.1338 | −0.4170 | −1.7703 | 0.2596 | 0.7160 | 0.6530 | 0.1338 | 0.8419 | 0.4327 | 0.4013 |
| GENE3740X | −1.5120 | −0.2122 | 2.0537 | −0.2122 | 1.1565 | 1.1910 | −1.5925 | −1.0749 | 0.4434 | −2.0871 | 0.9495 | 0.6274 | 0.1558 | 0.5699 |
| GENE3736X | −1.0718 | −0.9399 | 3.1475 | −1.5069 | 1.0379 | 0.5368 | −0.2411 | −0.3598 | 0.0753 | −0.2147 | 0.6951 | 0.9324 | −0.8081 | 0.3654 |
| GENE3682X | −0.9801 | −0.5265 | 0.5465 | 0.3485 | −1.2034 | 0.9282 | −1.0378 | 0.9570 | 0.5717 | −0.9981 | −0.4076 | 1.6339 | −1.2610 | 1.1010 |
| GENE3674X | −0.9609 | −0.4759 | −0.1600 | 0.4191 | −1.1565 | 0.7011 | −1.0324 | 0.7500 | 0.6071 | −1.2505 | −0.4571 | 1.4419 | −1.1640 | 1.1711 |
| GENE3673X | −0.9005 | −1.0086 | 0.4317 | 0.7475 | −1.4498 | 1.2319 | −0.7232 | 0.7215 | 0.9032 | −0.8616 | −0.4247 | 1.4655 | −1.3979 | 1.2060 |
| GENE3644X | −1.1211 | 0.6514 | 1.7303 | 0.5358 | 0.5743 | 0.4587 | −0.5624 | −1.2753 | −0.6973 | −1.4872 | 0.5165 | 0.7670 | −0.8321 | −0.1385 |
| GENE3472X | −0.5620 | 0.9628 | 0.8427 | −0.1418 | 1.5991 | 0.5546 | −0.4059 | −0.9342 | 0.0383 | −1.6546 | 0.2784 | 0.2544 | −0.1058 | 1.0588 |
| GENE2530X | −0.0835 | −0.2282 | 2.4848 | 0.0250 | −0.0655 | 0.7665 | −0.3006 | 0.7846 | 1.6709 | 0.1878 | 0.5857 | 1.0740 | 0.4772 | 0.6942 |
| GENE2287X | −0.3741 | 0.0024 | 1.1043 | 0.1860 | 0.1860 | 1.2328 | −1.0903 | 0.7645 | 1.6368 | −0.7414 | −0.2272 | 1.1318 | 0.0575 | 0.7921 |
| GENE2328X | −0.1288 | 0.4682 | 1.6062 | −0.7072 | 0.1324 | 0.1324 | −1.0616 | −0.0915 | 0.8413 | 0.4682 | −1.3974 | −0.0542 | −0.2408 | 0.0204 |
| GENE2417X | −0.9395 | 0.5096 | 0.4342 | −1.8301 | 1.4606 | 1.0682 | −0.1696 | 0.2983 | 0.1926 | 0.0417 | 0.4945 | 1.1134 | 0.1474 | 0.1323 |
| GENE2238X | 0.9909 | −0.3294 | −0.8129 | 1.7534 | 1.5302 | −2.0217 | −0.9431 | −0.0691 | −1.0547 | 1.5116 | −1.5940 | −0.5898 | 0.5446 | 1.1211 |
| GENE1971X | −0.9119 | −0.0072 | 2.4807 | −0.5161 | 0.4640 | 1.0294 | −1.4773 | −0.5349 | 0.7279 | −1.2888 | −0.8553 | 0.4263 | 0.4075 | −0.1768 |
| GENE3086X | 1.3005 | −1.0504 | −0.1077 | 0.5725 | 0.5606 | 0.0713 | 1.3363 | −0.5134 | −0.7163 | 2.7445 | −0.9550 | 0.3935 | 0.3339 | −0.2867 |
| GENE1009X | 1.0352 | 0.4600 | −1.0322 | 1.0196 | −0.4260 | 0.0870 | 0.5844 | −0.0840 | −0.5503 | 2.1232 | −0.1928 | −0.8612 | −0.1617 | 0.9263 |
| GENE1947X | 1.0880 | −0.4452 | 0.2940 | 0.0750 | 0.6225 | −2.2248 | −0.5547 | −0.2810 | −0.2810 | −0.0893 | −1.8963 | 0.2940 | 0.3214 | 0.7868 |
| GENE3190X | 0.9130 | −0.5824 | −1.3087 | −0.0376 | 0.5712 | −0.9455 | −0.1658 | 0.5605 | −0.1872 | −0.0910 | −0.0376 | −0.2406 | 1.1373 | 3.3376 |
| GENE3379X | 0.9185 | −0.4029 | −2.2407 | 0.9641 | −0.7218 | −0.9345 | −0.2054 | −0.4636 | −1.4660 | 2.0729 | −0.9648 | −1.8609 | −0.2054 | 0.5996 |
| GENE3184X | −1.3121 | 0.6890 | −0.8896 | 1.1892 | 0.2999 | −0.2337 | −0.2893 | 0.2777 | −0.6450 | 0.7112 | −0.2560 | −0.3782 | 0.4111 | 0.7446 |
| GENE3122X | −0.2819 | −0.9662 | −0.0766 | 0.5002 | 0.0505 | −0.2232 | −0.4578 | 0.1092 | 1.1552 | −0.2232 | −0.4383 | 0.4611 | 0.7739 | 1.1747 |
| GENE1099X | 0.8644 | −0.6805 | −1.8586 | 0.7005 | 0.2480 | −0.7039 | −0.5478 | −0.1655 | −0.3996 | −0.7585 | 0.1466 | −0.4230 | 0.4899 | 1.0282 |
| GENE3032X | 0.6600 | −0.8052 | −0.8478 | 0.8219 | 0.7622 | −1.3504 | −0.4645 | −0.0385 | −0.3282 | −0.7371 | 0.2767 | −0.5326 | 0.4130 | 1.0774 |
| GENE2675X | −0.1041 | −1.0945 | −1.8648 | 0.8963 | 0.9464 | −1.5147 | −0.0241 | 0.8363 | −0.7344 | −0.6743 | 0.7263 | −0.1341 | 0.6562 | 0.5162 |
| GENE22481X | −0.2042 | −0.9205 | −1.7274 | 0.9019 | 0.9563 | −1.2650 | −0.3946 | 0.6027 | −0.9477 | −0.6031 | 0.3035 | −0.0954 | 0.7115 | 0.8475 |
| GENE2878X | 0.4558 | −0.2223 | −1.1508 | 0.4036 | −0.1389 | −0.9526 | 1.3008 | −0.0032 | −0.8900 | 1.4365 | −0.5040 | −0.4101 | 2.1354 | 0.7375 |
| GENE2943X | 0.6388 | −0.2274 | −1.2512 | 1.1451 | 0.1776 | −0.9924 | 0.8188 | 0.0876 | −0.6212 | 2.0338 | −0.5424 | −0.1937 | 2.1013 | 0.6388 |
| GENE2977X | 1.4656 | −0.1900 | −0.0666 | 0.2059 | 0.4013 | −0.3134 | 0.9874 | 0.7406 | −0.5139 | 1.5941 | −0.7607 | −0.4059 | 0.8794 | 0.5710 |
| GENE3014X | 1.7123 | −0.6766 | −1.1738 | 1.6150 | −1.0225 | −0.0605 | 0.9880 | 1.3772 | −0.0064 | −0.0497 | −0.1470 | −0.2226 | 1.0853 | −0.0064 |
| GENE2006X | 1.0957 | −0.3782 | −1.2467 | −0.5492 | −0.4308 | 1.2931 | 0.5035 | 0.1614 | −0.3124 | 0.0429 | −0.1545 | −0.3782 | 0.8983 | −0.1281 |
| GENE1368X | −0.2260 | 0.2160 | −1.4968 | 0.2823 | −0.7564 | 0.3597 | −0.1265 | 1.2768 | −0.0602 | 0.3818 | 0.3155 | −0.3033 | 0.6249 | −0.0492 |
| GENE1184X | −0.0199 | 0.1558 | −1.0629 | 0.2327 | −0.7555 | 0.4522 | −0.0089 | 1.1000 | 0.0021 | 0.3754 | 0.2766 | −0.3712 | 0.5181 | −0.1846 |
| GENE1226X | −0.4983 | −0.4140 | −2.3779 | 0.5216 | 1.2717 | −0.3213 | 0.0411 | 0.4036 | 0.1254 | 2.4770 | −0.5826 | −1.2822 | 0.3867 | 0.4289 |
| GENE1228X | 1.3383 | −0.9973 | −1.4883 | 0.9311 | −0.0570 | −0.6499 | 0.9491 | −0.4044 | −0.7517 | 0.2723 | −1.3147 | −0.5781 | −1.1829 | 0.5059 |
| GENE1231X | −0.5801 | −0.1913 | −2.5674 | 0.1543 | 0.8743 | −0.8682 | −0.1049 | −0.7962 | −0.9258 | 0.8311 | −0.6521 | −1.6314 | 1.0327 | 1.2631 |
| GENE1246X | 0.0695 | −1.0162 | −2.6827 | 1.0206 | 0.5914 | −0.6290 | 0.1790 | −0.4523 | −0.6711 | 1.2226 | −1.5212 | −0.8226 | 1.4583 | 1.0206 |
| GENE1172X | 0.6118 | −1.3964 | −1.2171 | 1.1765 | 0.2083 | −0.3027 | 0.7014 | 0.0649 | −0.6882 | 1.9475 | −1.5578 | 0.0739 | 1.0690 | 0.3607 |
| GENE1164X | 2.1331 | −1.4831 | −1.6987 | 1.5360 | −0.4214 | −0.8693 | 1.1213 | 0.9388 | −0.3385 | 1.8843 | −0.8693 | −0.9191 | 1.7516 | −0.0067 |
| GENE3029X | 1.1569 | 0.0597 | −3.4516 | 1.4861 | −0.0135 | −0.0866 | 0.6997 | −0.3244 | 0.2608 | −0.3610 | −0.6353 | −1.1839 | 0.3157 | 0.1145 |
| GENE1027X | 1.1097 | −1.5512 | −1.9346 | 1.1097 | 0.2963 | −0.1104 | −0.7495 | −0.9818 | −0.9586 | −0.7727 | −0.8076 | −1.3304 | 0.6797 | −0.0871 |
| GENE1354X | 0.6660 | −0.5677 | 0.5538 | 1.0921 | 0.0828 | −0.0069 | 0.0603 | −0.8817 | 0.4865 | 1.3389 | −0.2312 | −1.3079 | 1.2267 | 0.5987 |
| GENE62X | 2.5246 | 0.7478 | −1.7550 | 0.5315 | 1.5512 | 0.5315 | −0.0246 | −0.4263 | −1.7705 | 0.2380 | −1.3997 | −0.5499 | 0.4852 | 0.8714 |
| GENE932X | −0.3542 | 0.9273 | 0.9273 | −0.6050 | 1.0388 | −0.4657 | −0.4935 | 0.7044 | 1.3731 | 0.1751 | 0.8437 | 2.1253 | −0.3542 | 0.5373 |
| GENE3611X | −0.5836 | −0.3891 | 0.2675 | −1.7265 | −0.8511 | 0.7052 | 0.0973 | −0.0243 | −0.2918 | 0.1459 | 0.9484 | −0.2675 | 0.7295 | 0.3161 |
| GENE3631X | −0.8746 | 0.0114 | 3.2187 | −0.0949 | 0.5430 | 0.4721 | −0.9632 | −0.7860 | −0.1126 | −0.2367 | 0.2949 | 0.6139 | −0.3430 | −0.4316 |
| GENE330X | −1.2586 | 0.1469 | 0.6520 | −0.3801 | 0.1689 | 0.6301 | −0.6217 | 0.4983 | 0.0152 | −0.0288 | 1.0254 | −0.1605 | 0.1689 | −0.2044 |
| GENE331X | −0.8855 | 0.5496 | 1.2585 | −1.0930 | 0.5323 | −1.3697 | −0.1074 | −1.2141 | 0.5496 | −0.8164 | −0.0729 | 0.8263 | −0.5224 | −0.1593 |
| GENE808X | 0.1648 | −0.6983 | −0.7813 | −0.1340 | 0.6461 | −1.3622 | −0.4327 | −0.7813 | −0.5987 | 0.0154 | −0.9638 | −0.1506 | 0.5797 | 0.4469 |
| GENE487X | 1.3843 | 1.3712 | −1.4128 | 1.0981 | 0.8769 | −1.9591 | 0.4996 | −0.0468 | −0.8143 | 1.0330 | −0.4631 | −0.9314 | −0.9054 | 0.5517 |
| GENE621X | 1.8500 | 1.4446 | −1.2623 | 0.7768 | 0.8364 | −1.5962 | 0.1209 | −0.0698 | −1.2385 | 1.2299 | −0.3918 | −0.7018 | −0.7138 | 0.8126 |
| GENE622X | 1.4051 | 1.5705 | −1.4906 | 0.5541 | 0.8968 | −1.5615 | 0.2704 | −0.3914 | −0.9351 | 0.8141 | −0.8642 | −1.0888 | −0.8287 | 0.8141 |
| GENE634X | −0.9764 | 0.7385 | 1.6582 | −1.2623 | −0.0568 | −0.3551 | 0.0302 | −0.5912 | −0.8770 | −1.1753 | 0.4403 | 0.6143 | −0.1562 | −0.2059 |
| GENE659X | −1.0919 | 0.4249 | 0.2082 | −1.3596 | 0.2974 | −0.2252 | 0.0297 | −0.9390 | −0.0977 | −1.2704 | 0.8965 | −0.3399 | 0.1062 | −0.0850 |
| GENE669X | −0.8278 | 0.4067 | 0.0934 | −1.3345 | 0.2224 | −0.4040 | 0.1579 | −0.3764 | 0.0566 | −0.9383 | 0.9318 | −0.1553 | 0.3606 | −0.1000 |
| GENE674X | −0.3922 | 0.5264 | −0.5367 | −0.6709 | 0.1755 | −0.0310 | 0.4541 | 0.0619 | 0.1135 | −0.7122 | 1.1560 | 0.0826 | 0.2787 | −0.4232 |
| GENE675X | −1.6557 | 0.3581 | 1.3386 | −2.0404 | −0.2453 | 0.7654 | 0.6975 | 0.0941 | 0.5693 | −0.1171 | −0.1397 | 0.8634 | 0.1469 | 0.3279 |
| GENE676X | −0.1988 | −0.0778 | −0.3198 | 0.2610 | 0.7814 | 0.7572 | −0.8039 | −0.1867 | 0.8056 | −0.0173 | −0.2351 | 0.9266 | −0.4892 | −1.2879 |
| GENE704X | −0.3770 | 0.0333 | 2.6244 | −0.7794 | −0.4575 | −0.4012 | −0.1035 | −0.2403 | 1.1679 | −0.6748 | −0.6104 | 0.4518 | −0.3127 | −1.1173 |
| GENE734X | −0.4844 | 0.0932 | 2.0981 | −0.9601 | −0.3995 | −0.3400 | −0.1191 | −0.4759 | 1.0872 | −0.6798 | −0.4929 | 0.2971 | −0.1191 | −0.6203 |
| GENE738X | −0.7216 | 0.1058 | 0.6496 | −1.1708 | 1.1224 | 0.3422 | −0.9344 | −1.1708 | 0.2477 | −1.2181 | −0.1779 | 1.3589 | −0.5325 | −0.7453 |
| GENE456X | −0.8475 | 0.1936 | 1.3418 | −0.0208 | 0.1170 | 0.2242 | −1.0771 | −0.8934 | 0.1170 | −0.9700 | −0.4648 | −0.8628 | 0.4385 | −0.3117 |
| GENE744X | −0.3044 | −0.1921 | 1.5886 | 0.1287 | −0.0959 | 0.3212 | −0.4649 | −0.2723 | 0.4175 | −0.4328 | −0.3205 | −0.1600 | 0.0966 | −0.6895 |
| GENE179X | 0.0345 | −0.4487 | 0.9089 | −0.6788 | −1.0699 | 0.1726 | 0.7248 | −0.4717 | 0.2416 | 0.3566 | −0.1265 | 0.6558 | 0.0575 | 0.0345 |
| GENE124X | −1.2150 | 0.2303 | 2.5199 | 0.0729 | −0.0129 | −0.6426 | −0.1704 | −0.0129 | 0.7026 | −0.9288 | 0.1302 | 0.8313 | −1.3009 | 0.1874 |
| GENE122X | −1.4265 | 0.4562 | 2.0049 | 0.0766 | 0.1222 | −0.2726 | −0.2422 | −0.0145 | 0.6840 | −1.0469 | 0.4410 | 0.3044 | −0.9254 | 0.2285 |
| GENE111X | −1.5857 | 0.5299 | 1.4521 | −0.1889 | 0.0959 | −0.4466 | −0.4737 | −0.8534 | 0.7333 | −1.6535 | 0.8689 | 0.3943 | −0.8399 | 0.4349 |
| GENE97X | −1.4927 | 1.1284 | 2.2424 | −0.9194 | 0.4240 | −0.5589 | −0.8866 | −0.4770 | 0.3748 | −0.0347 | 0.2602 | 0.2438 | −1.0996 | −0.3460 |
| GENE2645X | −0.2567 | 0.2983 | 1.8642 | −0.4549 | −0.9505 | −0.3360 | 0.1397 | 0.2190 | 1.6263 | −1.1289 | 1.0515 | 0.8334 | −0.1378 | 0.1992 |
| GENE3408X | 1.5515 | −0.1363 | 1.0562 | −0.8701 | 0.5058 | −0.8884 | 0.8177 | −0.1546 | 0.1389 | 2.8540 | −0.5215 | −0.3381 | −0.5215 | 0.3040 |
| GENE3854X | 1.4003 | 0.3319 | 0.1768 | −0.9605 | 0.7972 | −1.3052 | 0.4353 | −0.1506 | 0.0734 | 3.4338 | 0.1424 | −0.4263 | −0.0816 | 0.1768 |
| GENE1406X | 1.2709 | −0.0201 | −0.2427 | 0.5809 | −1.5783 | −1.9789 | 1.0705 | −0.3985 | −0.1092 | 0.2692 | −0.4876 | 0.4473 | 1.4712 | 0.1134 |
| GENE1401X | 1.1558 | 0.0547 | −0.4959 | 1.6749 | −0.0712 | −1.6756 | −0.8262 | 0.0075 | −0.8105 | 0.5738 | −1.5498 | −0.3543 | 1.4389 | 0.3693 |
| GENE3462X | −1.3172 | −0.3387 | 2.4462 | −0.2446 | −0.8656 | 0.5269 | −1.0161 | 0.5833 | −0.3387 | −0.9032 | 0.1694 | 1.1855 | −0.0188 | −0.3387 |
| GENE3173X | −1.1479 | −0.2676 | 2.6610 | 0.3926 | −0.9448 | 0.7142 | −0.2168 | 0.4603 | 0.8835 | −0.7416 | −0.0476 | 1.0358 | −1.1817 | −0.7755 |
| GENE3971X | 0.5571 | −0.0847 | −0.5224 | 0.5571 | 0.4696 | 0.4696 | 0.1139 | −1.6601 | −0.9891 | −0.1431 | −0.4348 | −0.9016 | 0.7613 | 0.9655 |
| GENE1756X | 0.7676 | −0.7601 | 0.8299 | 1.0949 | −0.7290 | −1.7266 | −0.3081 | −0.5419 | −0.1989 | 1.3132 | −1.2122 | −0.1210 | −1.0563 | 0.7364 |
| GENE1533X | −0.0992 | −0.4451 | 0.0662 | 1.0136 | −0.4451 | −1.9790 | −0.6406 | −0.8812 | −0.4451 | 0.0211 | −1.1519 | −0.8210 | −0.6706 | 1.1189 |
| GENE1757X | 1.0435 | 0.0925 | −0.0433 | 0.7854 | −0.2200 | −0.2471 | 0.2284 | −0.0705 | −0.5868 | −0.1928 | −0.5732 | −0.5460 | 0.1197 | 0.5408 |
| GENE3572X | −0.2343 | −0.1381 | 0.2465 | 0.0221 | −0.2984 | −0.3304 | 0.4708 | −0.7150 | −1.0356 | 1.8490 | −0.4907 | −1.1157 | 0.0221 | 0.6311 |
| GENE3571X | −0.3029 | −0.6058 | 2.3473 | −0.9541 | −0.6512 | 2.4079 | −0.2726 | −0.1060 | −0.0454 | 0.1212 | 0.7118 | 0.9238 | −0.2574 | −0.5603 |
| GENE385X | 0.2993 | 0.2292 | −0.2614 | −0.3549 | −0.4951 | 0.7431 | 0.1124 | −1.3127 | −0.1446 | −1.0557 | 0.6263 | 0.8366 | −1.2193 | −0.0979 |
| GENE1614X | 0.9780 | 0.2771 | 1.8700 | −0.4875 | −0.6998 | 0.6169 | −0.6149 | −0.7848 | 0.1072 | −0.2751 | 0.4045 | 0.9355 | −1.9741 | −0.7636 |
| GENE1623X | −0.8232 | 1.0462 | 1.6366 | −0.2722 | 0.3772 | 0.4559 | −0.6264 | −0.7445 | 1.3611 | −2.2991 | −0.1935 | 1.7153 | −1.0594 | 0.4362 |
| GENE1646X | −0.4711 | −0.2511 | 0.7077 | −0.7383 | −0.8169 | 0.1733 | 0.3462 | −0.4711 | 0.2676 | −0.7855 | 0.0632 | 0.3462 | −0.5183 | −0.7698 |
| GENE1660X | 2.5830 | 0.4392 | 0.1007 | 1.0598 | 0.6085 | −1.9302 | 0.4251 | 0.0584 | −0.9006 | 0.1289 | 0.5803 | −0.7596 | 1.5534 | 1.2008 |
| GENE1721X | 2.1035 | 0.3774 | 0.3409 | 0.8150 | 0.9852 | −2.0173 | 0.5841 | −0.2668 | −1.0448 | 0.5233 | 0.1343 | −0.4978 | 0.1586 | 0.4825 |
| GENE1573X | 0.5619 | −0.2361 | 0.1824 | 0.1337 | −0.1583 | 0.6008 | 0.3673 | −0.5086 | 0.4841 | −0.6546 | 0.5522 | −0.0707 | −0.6546 | −0.0512 |
| GENE1553X | −0.1660 | 0.7332 | 1.3021 | −0.2578 | 0.8066 | 1.1920 | −1.0836 | −1.2855 | 0.9534 | −1.0653 | −0.5698 | 0.0358 | −1.8544 | 0.0175 |
| GENE1773X | 0.1544 | −0.0483 | 0.7423 | −0.4131 | 0.4382 | 0.4787 | −0.2712 | −0.9604 | 1.3909 | −1.0009 | −0.5753 | 1.2085 | −1.4671 | 0.5801 |
| GENE913X | 1.0234 | 0.7291 | −0.2400 | −0.1682 | 1.2531 | −2.2284 | 0.3630 | −0.2112 | −0.8429 | 1.9925 | 0.3774 | −0.8142 | 0.0400 | 0.8942 |
| GENE3980X | 1.0738 | 0.6325 | −0.1799 | −0.2360 | 1.1999 | −1.9660 | 0.5905 | 0.1703 | −0.7403 | 1.8862 | 0.3734 | −0.8663 | −0.0118 | 0.7446 |
| GENE3X | −0.7588 | 0.4170 | 2.2246 | −0.4429 | 0.2766 | 0.9961 | 0.2064 | −1.1273 | 0.3117 | −0.8465 | −1.1624 | 0.2766 | −0.9167 | −0.8641 |
| RowNames | DLCL0031 | DLCL0032 | DLCL0033 | DLCL0034 | DLCL0036 | DLCL0037 | DLCL0039 | DLCL0040 | DLCL0041 | DLCL0042 | DLCL0048 | DLCL0049 | DLCL0051 | DLCL0052 |
| | | | | | OCT | | | | | | | | | |
| GENE3950X | 1.1111 | −0.7766 | −0.5316 | −1.3847 | 0.8298 | −1.2395 | 1.4560 | 0.5575 | −1.0489 | 2.1821 | −0.7403 | 0.6392 | −1.7024 | −2.8096 |
| GENE2531X | 1.0709 | −0.6452 | −0.8297 | −1.5309 | 0.7572 | −0.3684 | 1.6061 | 0.6557 | 0.7559 | 2.2981 | −0.7651 | 0.5635 | −2.0292 | −2.2322 |
| GENE918X | 0.9889 | −0.7984 | −0.8619 | −1.5061 | 0.8528 | −0.7349 | 1.5061 | 0.5807 | −0.7077 | 2.0686 | −1.2793 | 0.4355 | −2.0232 | −2.1684 |
| GENE3511X | −0.6954 | −0.2429 | −1.6794 | 0.4018 | −0.6162 | −0.9555 | 0.7864 | 2.4038 | 0.6846 | −0.5144 | 0.6054 | 1.1031 | −1.2043 | −1.4193 |
| GENE3496X | 1.0771 | −0.1580 | 0.9767 | −1.0216 | 0.7357 | −1.0116 | 0.6553 | 0.6654 | −1.3329 | 1.5088 | −0.9111 | 0.0328 | −1.6643 | −1.7446 |
| GENE3484X | 0.9644 | 0.1380 | 1.4603 | −0.9996 | 0.9158 | −0.7176 | 0.9644 | 0.7797 | −1.3107 | 1.3533 | −1.0288 | −0.3482 | −1.6899 | −1.8163 |
| GENE3789X | −0.2839 | −0.5622 | −1.2044 | −0.9475 | −0.2625 | −0.9261 | 0.9149 | 0.3583 | 0.4439 | 0.0158 | 0.3155 | 1.5785 | −1.6753 | −1.8037 |
| GENE3692X | 0.2311 | 0.3460 | −0.0878 | −1.1849 | −0.9170 | 1.8895 | 0.7159 | −1.0573 | −0.5725 | 0.0398 | −0.3174 | 0.0143 | −0.1133 | 2.3233 |
| GENE3752X | 0.8576 | −1.0464 | −0.5429 | −1.6601 | 0.7160 | −0.8733 | 0.8576 | 0.7632 | −0.1810 | 1.2667 | −0.3383 | 0.6688 | −0.9678 | −0.4957 |
| GENE3740X | 1.2830 | −0.1777 | −1.0864 | −0.7183 | 0.6389 | −0.2122 | 0.7769 | 0.1788 | −0.3273 | 1.7546 | 0.0512 | 0.0408 | −0.8103 | 0.8574 |
| GENE3736X | 1.1697 | 0.2731 | −1.0059 | −0.6367 | 0.4841 | −0.9267 | 1.2752 | 0.6423 | −0.4125 | 0.5105 | −0.0829 | 1.0774 | 0.6951 | −2.2716 |
| GENE3682X | 0.9102 | 0.2837 | −1.0198 | −0.4833 | 1.8896 | −0.2600 | 1.8824 | 0.7158 | 0.4889 | 0.5681 | −0.9981 | 0.6689 | −1.1782 | −1.3402 |
| GENE3674X | 1.3065 | 0.6221 | −1.5099 | −0.0998 | 1.8781 | −0.3781 | 1.4757 | 0.4379 | 0.5695 | 0.9380 | −0.9985 | 0.7011 | −1.2693 | −1.4610 |
| GENE3673X | 0.9248 | 0.8859 | −1.2379 | −0.3512 | 1.1324 | −0.1133 | 1.2579 | 1.0676 | 1.3401 | 1.2016 | −0.3166 | 0.9075 | −1.3244 | −1.6575 |
| GENE3644X | 0.3239 | −0.5817 | −0.5046 | −1.0826 | −0.7165 | −0.0615 | 1.9615 | 1.4028 | 0.6707 | 2.0000 | −0.3890 | 0.8633 | −0.2156 | −1.7376 |
| GENE3472X | 0.6146 | −0.2979 | −0.9462 | −1.4385 | 0.6506 | −1.1383 | 0.8908 | 0.4465 | −1.2704 | 2.8718 | −0.0457 | 0.2054 | −0.9702 | −1.1023 |
| GENE2530X | 0.4952 | −0.6442 | −1.1868 | −1.5124 | 1.3815 | −0.6623 | 1.1825 | 0.7304 | 0.6038 | 0.1516 | −1.8199 | 1.7794 | −2.4891 | −1.2592 |
| GENE2287X | 0.5717 | −1.1270 | −1.6504 | −1.4392 | 0.7921 | −0.0986 | 0.8013 | 1.2053 | 0.4707 | 1.8113 | −1.5402 | 1.5909 | −2.6513 | −0.6220 |
| GENE2328X | 1.3077 | −0.5392 | −2.3862 | −0.6885 | 0.3376 | −0.6325 | 1.0652 | 1.2704 | −0.0915 | 1.3823 | −0.4833 | 1.7741 | 0.7294 | −0.8751 |
| GENE2417X | 0.3134 | 0.0115 | −0.4413 | −1.0904 | −0.9848 | −1.1357 | 0.7059 | −0.4263 | −0.6527 | 0.5247 | −0.6376 | 0.0417 | −1.1206 | −1.5131 |
| GENE2238X | −0.1063 | 1.3071 | −0.8501 | 1.2141 | −1.7986 | 0.8794 | 0.7120 | −0.9803 | −1.3336 | 0.2285 | −0.7571 | −0.4038 | −0.2736 | 0.9537 |
| GENE1971X | 1.0294 | 0.0682 | −1.4396 | −0.5538 | 0.9917 | −0.4030 | 0.0494 | −1.0438 | −0.4972 | 2.8577 | −0.1203 | 0.7844 | −0.8365 | −1.4208 |
| GENE3086X | 0.2742 | −0.1077 | 3.3650 | −0.2748 | −1.0624 | 0.5129 | −1.2414 | −1.4562 | 0.5606 | −0.4299 | −0.4299 | −0.7998 | 0.7993 | −0.3583 |
| GENE1009X | −1.9182 | −0.5348 | −1.5607 | 0.7398 | −1.0944 | 2.2476 | −1.1099 | −0.3949 | −1.7161 | −0.5037 | 0.5688 | −0.3638 | 1.5015 | 1.2683 |
| GENE1947X | −1.8415 | 0.6773 | −1.1297 | 0.9237 | −0.5274 | 1.2249 | −0.5821 | −1.6499 | 0.9511 | 0.7047 | −1.5404 | −1.1297 | 0.7868 | 1.0058 |
| GENE3190X | −0.5076 | 1.3402 | −0.4435 | −0.2833 | −0.9242 | −1.3087 | −0.4008 | −0.7105 | −0.5396 | −1.1592 | −0.7212 | −0.1765 | −0.9562 | 1.8209 |
| GENE3379X | −0.0080 | 1.0552 | −1.5420 | 1.1312 | −0.1447 | 0.5085 | −0.9800 | −1.3597 | 0.2047 | 0.2654 | −0.6762 | −0.0991 | 0.2502 | 1.9969 |
| GENE3184X | −1.7456 | 0.4889 | −0.3894 | 0.9113 | −1.7678 | 1.6228 | −0.5561 | −1.2565 | −0.6450 | −0.2782 | −0.5005 | −1.2342 | 0.3777 | 2.1342 |
| GENE3122X | −0.0766 | −0.5263 | −0.4481 | 1.8590 | −0.0668 | 0.8228 | −1.2203 | −0.0472 | −3.2243 | −2.2663 | −1.1519 | −0.0179 | 0.2167 | 1.4484 |
| GENE1099X | −0.6961 | 1.1062 | −0.7195 | 1.1609 | −1.7104 | 2.0269 | −1.4997 | 0.8566 | 1.6368 | −2.0069 | 0.5211 | −1.2734 | 1.0126 | 1.4027 |
| GENE3032X | −0.6860 | 1.1285 | −0.3622 | 0.7111 | −2.0916 | 2.0060 | −1.9638 | 0.0807 | 1.6226 | −1.4015 | 0.5152 | −0.8393 | 1.0604 | 1.9037 |
| GENE2675X | −1.3446 | 0.4061 | 0.4862 | 0.5262 | −1.1345 | 0.0960 | −0.3442 | −1.4247 | 1.5366 | −1.2946 | 0.2861 | 0.4361 | −0.2241 | 1.5166 |
| GENE2481X | −1.1199 | 0.5030 | 0.8112 | 0.6934 | −0.9386 | 0.2400 | −0.4127 | −1.6367 | 1.4731 | −1.4735 | −0.6666 | −0.2677 | 0.0043 | 1.7542 |
| GENE2878X | −1.0986 | 1.7599 | −0.7439 | 0.3932 | −1.1091 | 2.5319 | −0.9735 | −0.8796 | −1.2447 | −0.1180 | −0.9526 | −0.8065 | −0.5562 | 0.7062 |
| GENE2943X | −0.8012 | 1.2913 | −0.7112 | 0.2676 | −1.2849 | 0.9763 | −1.2849 | −0.2049 | −0.9362 | −1.1049 | −0.9812 | −1.4199 | −1.0712 | 0.2113 |
| GENE2977X | −1.0743 | 0.8229 | −1.0435 | 1.7843 | −1.3468 | 1.6250 | −0.8944 | 0.4116 | −1.0486 | −1.5525 | −0.6424 | −0.7144 | −1.1463 | 1.6095 |
| GENE3014X | −1.2819 | 0.3395 | −0.8063 | 0.2530 | 0.7286 | 1.8852 | −0.0172 | −0.5361 | −0.1253 | −1.5306 | −1.0874 | −0.5793 | −1.1955 | 0.6637 |
| GENE2006X | −0.7466 | 0.4509 | 0.3587 | 1.7800 | −0.1150 | 2.9775 | −0.4177 | −0.8519 | −0.7335 | −1.1941 | −1.6941 | −1.6941 | −0.0097 | 0.5167 |
| GENE1368X | −1.2316 | 0.4370 | −0.0934 | 1.6967 | −1.5189 | 1.0448 | −0.6127 | −0.3807 | −2.9443 | 0.4702 | −1.1211 | −1.2095 | −0.6901 | 1.8846 |
| GENE1184X | −1.1398 | 0.5181 | −0.0967 | 1.3965 | −1.5680 | 1.7698 | −0.6018 | −0.2724 | −3.3027 | 0.3754 | −1.2276 | −1.1727 | −0.8433 | 1.7039 |
| GENE1226X | −0.1106 | 0.4289 | 1.0273 | 1.2380 | −1.2569 | 0.9430 | −1.1726 | −0.8692 | 0.1254 | −1.2737 | −1.2063 | −0.0179 | 0.3867 | 0.6733 |
| GENE1228X | −0.8835 | 0.2664 | 1.1766 | −0.4762 | −0.8416 | 1.3563 | −0.6679 | −0.6559 | −1.1410 | −1.0452 | 0.0687 | −0.7577 | 2.4403 | −0.5182 |
| GENE1231X | 0.7303 | 0.9895 | 1.6232 | 0.9175 | −1.0410 | 0.3559 | −0.3209 | −1.1130 | −1.1130 | 0.9895 | 0.1543 | −0.4361 | 1.0471 | 1.6664 |
| GENE1246X | −0.6375 | 1.0459 | 1.2479 | 1.0879 | −0.2587 | 0.6334 | 0.2968 | −1.0751 | −0.5533 | 0.7176 | −0.2250 | −0.6030 | 0.9617 | 1.3825 |
| GENE1172X | −1.3605 | 0.5400 | 0.9614 | 0.4145 | −0.1503 | 1.5262 | 0.2442 | −0.8854 | −0.2399 | 0.1904 | −0.2847 | −0.1323 | 0.2532 | 1.4007 |
| GENE1164X | −1.3006 | 0.1094 | 0.8061 | 0.1758 | −0.0233 | 1.5028 | 0.4743 | −0.8693 | 0.4246 | −0.3717 | −0.5873 | 0.4578 | −0.3717 | −0.7366 |
| GENE3029X | −0.5621 | 0.0779 | 0.0231 | 1.7604 | 0.3705 | 0.5169 | −1.0010 | −1.5131 | 0.8277 | −1.3851 | −1.4034 | −0.2330 | −0.4341 | 1.1935 |
| GENE1027X | 0.2382 | −0.5635 | 1.7022 | 0.7611 | −1.2259 | 1.2375 | −0.9237 | −0.0407 | −0.7959 | −1.1561 | −0.0174 | −0.1104 | 2.2832 | −0.1104 |
| GENE1354X | −1.1284 | 0.5090 | 0.5987 | 1.7650 | 0.1276 | 0.8230 | −0.5228 | −0.7247 | −2.1602 | −3.9322 | −1.0836 | 0.5090 | −0.2088 | 0.7781 |
| GENE62X | −1.3688 | 0.1299 | 0.1453 | 0.5006 | −0.9980 | 1.4585 | 0.0681 | −1.3070 | −0.8898 | −0.5036 | 0.2534 | 0.3925 | −0.1946 | 1.0105 |
| GENE932X | −0.3264 | −0.5492 | −1.9143 | −0.9950 | 1.6795 | 0.6209 | 0.4259 | 0.2587 | 0.2308 | 2.0138 | 0.5652 | 1.4845 | −1.8029 | −0.4099 |
| GENE3611X | 1.5563 | −0.7782 | −0.4620 | 0.8025 | 0.5350 | 0.3891 | 0.4620 | 0.7538 | 0.6809 | 2.9181 | 0.2432 | −0.0730 | −0.3161 | −0.8268 |
| GENE3631X | 0.0646 | −0.9455 | −1.9201 | −0.2898 | 0.4544 | −0.2721 | 0.6316 | 0.1000 | 2.2973 | 0.9683 | −0.3607 | 1.5530 | 1.3227 | −1.3708 |
| GENE330X | −0.1825 | −0.0727 | −1.3025 | −0.9950 | −0.4240 | −0.1605 | −0.0946 | −0.0068 | 1.3987 | 2.6065 | 0.7179 | 2.1893 | 0.0591 | −0.1386 |
| GENE331X | −0.2804 | −0.1939 | −0.2112 | −0.1420 | 0.9300 | −0.8164 | 0.9127 | 0.6015 | −0.0037 | 0.8781 | 0.2557 | 0.1865 | 1.8637 | −1.7847 |
| GENE808X | −0.6983 | 1.8411 | −0.0676 | −0.3165 | −0.7979 | 0.0984 | −1.4286 | −1.5779 | −0.9804 | 0.0486 | −0.3331 | −0.4825 | 3.9324 | 0.9117 |
| GENE487X | −1.6860 | −0.2289 | 0.7598 | 1.7095 | −1.0615 | 1.0720 | 0.0833 | −0.7883 | −0.2939 | −2.1543 | 0.7078 | 0.5517 | −0.6842 | 0.1484 |
| GENE621X | −1.5843 | −0.7853 | 1.1226 | 1.7069 | −1.2981 | 0.8245 | 0.0733 | −1.0954 | −0.2487 | −1.8705 | 0.8603 | 0.3833 | −0.6422 | 0.4310 |
| GENE622X | −1.6679 | −0.3205 | 1.3342 | 1.7951 | −1.2306 | 0.2468 | 0.5659 | −1.0297 | −0.1432 | −1.8452 | 0.6368 | 0.6014 | −0.1078 | 0.9678 |
| GENE634X | 0.8628 | 0.0302 | 0.0799 | −0.0941 | 0.4900 | −0.8149 | 0.6267 | 0.2663 | −2.0576 | 3.1122 | 1.4966 | 0.0178 | −0.4048 | −1.6227 |
| GENE659X | 1.0877 | 0.6033 | 0.4376 | −1.0919 | 0.6416 | −0.7478 | 0.3102 | −0.4801 | −1.9459 | 1.5975 | 0.8582 | −0.6840 | 0.6925 | −1.4998 |
| GENE669X | 1.1068 | 0.6738 | 0.3606 | −1.5464 | 0.3422 | −0.6528 | 0.5817 | −0.1829 | −2.0991 | 1.4016 | 0.6278 | −0.4961 | 0.0290 | −2.2004 |
| GENE674X | 0.8670 | 0.5057 | 0.1755 | −1.8475 | 0.2993 | −0.7431 | 0.4645 | −0.2684 | −2.1262 | 1.3005 | 0.9599 | −0.4438 | −0.2684 | −2.3635 |
| GENE675X | 1.2028 | −0.1699 | −0.9392 | −0.3358 | 1.4366 | −0.8638 | 0.4712 | 0.5843 | −0.4489 | 1.5497 | 0.8483 | 0.2977 | 0.0262 | −2.7342 |
| GENE676X | 0.0674 | −0.4408 | −0.4408 | −0.2230 | −0.0657 | −1.1185 | −1.0822 | 1.7374 | −1.3969 | 0.5273 | 1.0960 | 0.9266 | −1.5179 | −1.2516 |
| GENE704X | 1.0633 | −0.1035 | −0.8277 | −1.1093 | 1.2967 | 0.2506 | 0.9587 | 0.9185 | 3.1152 | 0.8219 | 0.8058 | 1.1438 | −1.3668 | −1.6323 |
| GENE734X | 1.2316 | −0.1956 | −0.8072 | −0.8242 | 1.2061 | −0.7902 | 0.9597 | 1.0277 | 3.2704 | 1.0956 | 1.0532 | 1.3250 | −1.7332 | −1.0536 |
| GENE738X | 1.2406 | −0.2488 | −0.1070 | −0.7216 | 0.6496 | −0.0124 | 2.0445 | 0.6260 | −1.1472 | 1.3589 | 0.1768 | 0.6496 | −0.8399 | −0.7216 |
| GENE456X | 1.9082 | −0.5413 | −1.5517 | −0.8934 | 1.2499 | −0.8934 | 1.1887 | 0.7753 | 2.0766 | 2.3063 | 0.1017 | 0.4844 | −1.2762 | 0.2657 |
| GENE744X | 1.6047 | −0.6253 | −2.4221 | −1.1226 | 1.1394 | −0.8820 | 1.7811 | 0.8025 | 2.1982 | 1.7170 | −0.0477 | 0.1929 | −1.3312 | −0.5290 |
| GENE179X | 0.6788 | −0.3796 | −0.3106 | 0.2646 | 1.0929 | 1.1389 | 1.6681 | 1.3690 | 0.7018 | 1.6221 | 1.7602 | 0.4717 | −0.4027 | −0.9779 |
| GENE124X | 0.6024 | −0.7571 | −0.0416 | −0.0416 | 0.3305 | −0.8000 | 0.9172 | 1.7615 | 1.1032 | 1.5755 | 0.4020 | 0.1731 | 0.4593 | −2.2024 |
| GENE122X | 0.5169 | −0.8647 | −0.2878 | 0.2892 | 0.3044 | −1.0621 | 0.8206 | 1.9593 | 1.0787 | 1.2002 | 1.0332 | 0.5018 | 0.5929 | −2.2160 |
| GENE111X | 0.7604 | −1.1111 | −0.2025 | 0.6926 | 0.7197 | −1.1518 | 0.5027 | 1.1944 | 1.1808 | 1.8047 | 0.6655 | −0.2296 | 0.8553 | −2.0875 |
| GENE97X | −0.1002 | −0.0308 | −0.8374 | 0.5550 | −0.4934 | −0.6572 | −0.0183 | 0.9482 | −0.3951 | 3.1435 | 1.8820 | 0.4404 | 1.0629 | −0.3624 |
| GENE2645X | −0.2171 | −1.4064 | −0.2171 | −1.5055 | 0.5361 | 0.4370 | −1.1685 | 1.9434 | −1.2676 | 0.2190 | 1.2893 | 0.9325 | −1.7830 | −0.7919 |
| GENE3408X | −1.6589 | 0.6159 | 0.6709 | 0.6709 | 1.6983 | −0.2464 | −0.5215 | −0.8884 | −1.4205 | −0.1730 | −0.8517 | −1.1269 | 1.3313 | 0.8544 |
| GENE3854X | −1.2879 | 0.5043 | 0.1500 | 0.7800 | 0.9695 | −0.4263 | −0.9433 | −1.4775 | −0.5814 | 0.5387 | −0.6331 | −0.5125 | 1.2453 | 0.8317 |
| GENE1406X | −0.5098 | 0.8034 | 1.9386 | 0.8925 | 0.4028 | 2.2058 | −1.0663 | −0.7102 | 0.6031 | −1.3334 | −1.2666 | −1.1108 | −0.1537 | 2.0054 |
| GENE1401X | −0.3700 | 0.2434 | −0.3858 | 0.5109 | −0.8891 | 0.0075 | −1.3925 | −0.3071 | 0.2120 | −0.0240 | −0.0554 | −0.7318 | −0.5903 | 2.4299 |
| GENE3462X | 2.2580 | −0.6962 | −1.8064 | 0.0941 | 0.9408 | −1.0161 | −0.3011 | 0.5833 | 0.8279 | 2.6908 | 0.0188 | −0.3763 | −0.0941 | 0.6774 |
| GENE3173X | 0.4434 | −0.5046 | −0.5893 | 1.5268 | 1.8484 | −0.4708 | 0.3418 | 2.1023 | 0.8158 | 1.0189 | −0.2507 | −1.1140 | −0.9786 | −1.4865 |
| GENE3971X | −1.6310 | −0.1431 | 1.1114 | 1.3740 | −2.2436 | 1.6365 | 0.9072 | −0.6099 | −2.0394 | 1.3740 | −0.4057 | −0.9308 | 0.0903 | 1.4907 |
| GENE1756X | −0.3081 | 0.3311 | 1.7340 | 0.5025 | −0.0119 | 1.3443 | −0.4016 | −0.2301 | 1.1105 | −1.3525 | 0.5649 | −0.6510 | 0.5493 | 1.3911 |
| GENE1533X | 0.1114 | 0.5324 | 1.8558 | 1.1941 | −0.2496 | 0.2166 | −0.8661 | −0.5202 | 0.8181 | −0.6105 | 0.9685 | −0.7759 | 1.4046 | 2.0814 |
| GENE1757X | −0.4509 | 0.2555 | 0.2827 | 1.7500 | −1.1302 | 0.3099 | −0.7498 | −1.1030 | −2.3529 | −1.3204 | 0.2555 | −0.1520 | −0.8721 | 3.4890 |
| GENE3572X | −1.2920 | 0.5029 | 1.6247 | 1.4164 | −0.3785 | 0.2305 | −1.2920 | −1.4843 | −1.1477 | −0.7631 | 0.4869 | −0.7952 | 3.0670 | −0.3625 |
| GENE3571X | 0.2877 | −0.3029 | 0.3483 | 1.0298 | 2.8319 | −0.4543 | 0.8329 | −0.3635 | −0.5906 | 1.4841 | −0.5603 | 0.4240 | −0.9238 | −1.4084 |
| GENE385X | −0.3549 | −0.7287 | −1.4996 | −0.1213 | 2.7289 | −2.2939 | 0.7665 | 1.1403 | −0.5184 | 0.5329 | 1.1403 | 0.6497 | −0.0979 | 2.1215 |
| GENE1614X | −0.8697 | −0.8697 | −1.8255 | −0.6574 | 2.4646 | 0.4045 | 0.6382 | 1.0842 | −0.4450 | −0.2963 | 0.6594 | 0.2559 | −0.3388 | 1.6363 |
| GENE1623X | 0.0230 | −0.6658 | −0.3313 | −0.9216 | 1.0462 | −2.8304 | −0.5871 | 1.3021 | −0.4100 | 0.8495 | 0.3968 | −0.3509 | −1.4332 | 0.4165 |
| GENE1646X | −0.0153 | −0.6598 | 0.4876 | −0.0468 | 3.8354 | 0.2676 | 0.9906 | 2.5623 | 0.0947 | −0.8484 | −0.7698 | −0.2825 | 0.2676 | −1.0055 |
| GENE1660X | −0.8301 | 0.4392 | −0.0685 | −0.3083 | −0.3365 | −0.4352 | 0.2136 | 0.4110 | −1.7469 | −2.5790 | −0.8160 | 0.2277 | 0.8482 | 0.8200 |
| GENE1721X | −0.8868 | 0.2802 | 0.4747 | −0.6801 | −0.0845 | 1.8847 | 0.3166 | 0.8272 | −1.3366 | −3.0870 | −0.8625 | 0.2194 | 0.8515 | 0.7664 |
| GENE1573X | 0.6787 | −1.2191 | 0.8200 | −1.5986 | 0.0753 | 1.1166 | 1.0485 | 2.8976 | 1.5838 | 0.1337 | −0.5378 | 1.4573 | −1.8127 | −2.6010 |
| GENE1553X | 1.0452 | −0.7350 | −0.7533 | −1.1571 | 0.4029 | −0.3496 | 0.4212 | 1.4306 | −1.0836 | 1.6692 | 0.8984 | 0.3662 | −1.9462 | −0.3312 |
| GENE1773X | 1.1679 | −0.4739 | −0.8388 | 1.3455 | 0.6814 | 0.8436 | 0.8639 | 1.7963 | −1.1834 | 1.4720 | 0.1139 | 0.3977 | −2.2779 | −0.4131 |
| GENE913X | −0.6922 | 0.9014 | 1.1957 | 0.2195 | −1.8551 | 1.0880 | 0.5927 | −0.8788 | −1.7761 | −1.8048 | −0.2687 | −1.3526 | 0.0974 | 0.5999 |
| GENE3980X | −0.8943 | 0.8917 | 0.9337 | 0.3314 | −1.8189 | 1.1788 | 0.4574 | −0.9854 | −2.0990 | −1.8189 | −0.1729 | −1.3986 | 0.1422 | 0.8567 |
| GENE3X | 0.0836 | −0.6359 | −0.8992 | −0.9869 | 0.6276 | 0.7329 | 1.2594 | 1.5928 | −0.6008 | 0.9786 | −0.6008 | 0.8206 | −1.2151 | −1.4783 |

In the claims which follow and in the preceding description of the invention, except where the context requires otherwise due to express language or necessary implication, the word “comprising” is used in the sense of “including”, i.e. the features specified may be associated with further features in various embodiments of the invention.

It is to be understood that a reference herein to a prior art document does not constitute an admission that the document forms part of the common general knowledge in the art in Australia or in any other country.

1. A method for identifying components of a system from data generated from the system, which exhibit a response pattern associated with a test condition applied to the system, comprising the steps of:
specifying design factors to specify a response pattern for the test condition;
identifying a linear combination of components from the input data which correlate with the response pattern.

2. The method of claim 1 wherein the design factors are specified as a matrix of design factors.

3. A method according to claim 1 wherein the linear combination of components is in the form of:
Y=a₁X₁+a₂X₂+a₃X₃ . . . +aₙXₙ
wherein Y is the linear combination, a₁-aₙ are component weights generated from the method and X₁-Xₙ are data values for components of the system.

4. A method of claim 3 further comprising the step of:
establishing the weights of the components by maximising the value λ of a test for significance of a linear regression of the linear combination of the components on the design factors.

5. A method of claim 4, wherein the test for significance of the linear regression is performed by calculating
λ=a^{t}Ba/a^{t}Wa
where W is a within groups matrix, and B is a between groups matrix
wherein B=XPX^{T} and W=X(I−P)X^{T}, wherein X is a data matrix having n rows of components and k columns of test conditions, P=T(T^{T}T)⁻¹T^{T} wherein T is a matrix of k rows of design factors and r columns, and a is a weight matrix for the linear combination y^{T}=a^{T}X.

6. A method of claim 5, wherein the maximum value of,% is obtained by solving the equation
(B−λW)a=0, (1)
to determine a and λ.

7. A method of claim 6, further comprising the steps of:
substituting X(I−P)X^{T}+σ²I for the within groups matrix W; and
solving Equation 1 to identify the linear combination.

8. A method of claim 6 further comprising the step of solving Equation 1 without requiring calculation of B or W by using the generalised singular value decomposition.

9. A method of claim 6, further comprising the step of generating at least one intermediate matrix in solving Equation 1, wherein the size of each intermediate matrix is no greater than the size of the data matrix X.

10. A method according to claim 6, further comprising the steps of:
a) establishing a model covariance matrix V
(b) substituting V for the within groups matrix W in Equation 1; and
(c) solving Equation 1 to identify the linear combination using the matrix V substituted for the within groups matrix W.

11. A method according to claim 10, further comprising the steps of:
establishing a model of the data generated from the system; and
estimating the covariance matrix in the model given the available data.

12. A method according to claim 10, wherein the covariance matrix V is of the form
VΛΦΛ+σ²=I
wherein Λ is an n by s matrix of factor loadings, Φ is a diagonal s by s matrix and σ² is a variance parameter;

13. A method according to claim 11, further comprising the steps of:
establish a model for the residuals of the regression of the input data on the design factors; and
estimating parameters for the model.

14. A method for identifying components of a system from data generated from the system, which exhibit response patterns to a test condition applied to the system, comprising the steps of:
specifying design factors to specify a response pattern for a test condition;
establishing a model for the residuals of a regression of the input data on the design factors;
estimating parameters for the model; and
computing a linear combination of components using the model and the estimated parameters.

15. A method of claim 14, wherein the linear combination of components is in the form of:
Y=a₁X₁+a₂X₂+a₃X₃ . . . . +aₙXₙ
wherein Y is the linear combination, a₁-aₙ are component weights generated from the method and X₁-Xₙ are data values for components of the system; and wherein the method further comprising the step of:
establishing the weights of the components by maximising the value λ of a test for significance of a linear regression of the linear combination of the components on the design factors, wherein the maximum value of λ is obtained by solving the equation
(B−λW)a=0, (1)
to determine a and λ
wherein B=XPX^{T} and W=X(I−P)X^{T}, wherein X is a data matrix having n rows of components and k columns of test conditions, P=T(T^{T}T)⁻¹T^{T} wherein T is a matrix of k rows of design factors and r columns, and a is a weight matrix for the linear combination y^{T}=a^{T}X.

16. A method of claim 13, further comprising the steps of:
modelling the data using a multivariate normal distribution which is specified by mean model and variance model to establish the data model
using the data model to model for the residuals
estimating the parameters in the mean model and the variance model; and
establishing the covariance matrix from the data model in the form of:
V²=I
wherein Λ is an n by s matrix of factor loadings, is a diagonal s by s matrix and σ² is a variance parameter;

17. The method of claim 12, wherein the estimate of Λ may be computed from the left singular vectors of R, wherein
R=X−\hat{B}T^{T}, and
\hat{B}=X^{T}T(T^{T}T)⁻¹

18. The method of claim 17 wherein the estimate of σ² is computed from the equation:
$$s {σ}^{2} = 1 / ( k ( n - s ) ) \{ tr \{ {RR}^{T} \} - {\sum }_{I = 1}^{S} {δ}_{ii} \} ,$$
wherein the δᵢᵢ are the squares of the singular values of R.

19. The method of claim 18 wherein the estimate of Φ is computed from the equation:
Φᵢᵢ+σ²δᵢᵢ/k

20. A method of claim 19, wherein the linear combination is identified from the equation:
a=λ^{-1/2}Xpu (2)
wherein a is the vector of weights for the linear combination y^{T}=a^{T}X, P=T(T^{T}T)⁻¹T^{T}, u is an eigenvector of P(XV⁻¹X^{T})P or equivalently a right singular vector of V^{-1/2}XP;
and X is an nxk data matrix of data generated from a method applied to a system, wherein the data is from n components and k test conditions.

21. A method of claim 12, wherein the number of factors s in the variance model V is computed using the Bayesian method whereby the number of factors is chosen to maximise
$$\begin{matrix}log P ( R \vert s ) = log P ( u ) - 0.5 n {\sum }_{j = 1}^{s} log ( {λ}_{j} ) - & \\ 0.5 n ( k - s ) log ( v ) + 0.5 ( m + s ) log ( 2 π ) - & \\ 0.5 log det ( {A}_{z} ) - 0.5 s log ( n ) & \end{matrix}$$
where m=ks−s(s+1)/2,
$$\begin{matrix}log P ( u ) = - s log ( 2 ) + {\sum }_{i = 1}^{s} \{ log ( Γ ( ( k - i + 1 ) / 2 ) ) - & \\ 0.5 ( k - i + 1 ) log ( π ) \} & \\ v = ( {\sum }_{j = s + 1}^{k} {λ}_{j} ) / ( k - s ) & \\ and & \\ log det ( {A}_{z} ) = {\sum }_{i = 1}^{s} {\sum }_{j = i + 1}^{k} log ( ( {\hat{λ}}_{j}^{- 1} - {\hat{λ}}_{i}^{- 1} ) ( {λ}_{i} - {λ}_{j} ) n ) & \\ where & \\ {\hat{λ}}_{j} = \{ \begin{matrix}{λ}_{j} , for j \leq k \\ v , otherwise .\end{matrix} & \end{matrix}$$
and the λⱼ are the squared singular values of the matrix R.

22. A method for estimating missing values from the results of the method of claim 16, the method comprising the steps of:
(a) estimating initial values of B, Λ, Φ and σ by replacing missing values with simple estimates and calculating maximum likelihood estimates assuming the data was complete;
(b) computing E{X|o₁, . . . oₖ} and E{RR^{T}|o₁, . . . , oₖ} the expected values of the data array and the residual matrix under the model given the observed data and current parameter estimates;
(c) substitute quantities from (b) into likelihood equations assuming the data is complete to obtain estimates of B, Λ, Φ and σ²;
(d) repeat steps (b) and (c) until convergence.

23. A method of claim 1 comprising the further step of:
determining the significance of each weight of the linear combination; and
setting non-significant weights to zero.

24. A method of claim 23 wherein the significance of the weights of the linear combination is determined by a permutation test comprising the steps of:
a) randomising the data for the components of a linear combination;
b) computing the weights and eigenvalues from the randomised data;
c) repeating steps a) and b) a plurality of times;
d) determining a distribution for the weights and eigenvalues computed from the randomised data;
e) determining the position of weights and eigenvalues computed from non-randomised data relative to the distribution of the weights and eigenvalues computed from randomised data; and
f) determining the significance of each weight computed from the non-randomised data.

25. A method of claim 1 wherein the significance of the overall linear combination is determined by a permutation test comprising the steps of:
(a) randomising the data for the components of a linear combination;
(b) computing the weights and eigenvalues from the randomised data, and from these computing the squared multiple correlation coefficient of the linear combination with the columns of the design basis;
(c) repeating steps a) and b) a plurality of times;
(d) determining a distribution for squared multiple correlation coefficient computed from the randomised data;
(e) determining the position of the squared multiple correlation coefficient from non-randomised data relative to the distribution of the squared multiple correlation coefficient computed from randomised data; and estimating the significance of the squared multiple correlation coefficient computed from the non-randomised data.

26. A method of claim 1 wherein the response pattern as specified by the design factors is derived from known data.

27. A method of claim 1 wherein the response pattern as specified by the design factors is derived from the input array data.

28. A method of claim 1 wherein the response pattern as specified by the design factors is selected to identify an arbitrary response pattern.

29. A method of claim 1 wherein the data is generated from the system using a method selected from the group consisting of DNA array analysis, DNA microarray analysis, RNA array analysis, RNA microarray analysis, DNA microchip analysis, RNA microchip analysis, protein microchip analysis, carbohydrate analysis, DNA electrophoresis, RNA electrophoresis, one dimensional or two dimensional protein electrophoresis, proteomics, antibody array analysis.

30. A computer program which includes instructions arranged to control a computing device to identify linear combinations of components from input data which correlate with a response pattern in a defined matrix of design factors specifying types of response patterns for a set of test conditions in a system.

31. A computer readable medium providing the computer medium of claim 30.

32. A computer program which includes instructions arranged to control a computing device, in a method of identifying components from a system which exhibit a response pattern to a test condition applied to the system, and wherein a matrix of design factors specifying the response patterns for the test conditions is defined, to formulate a model for the residuals of a regression of the input data on the design factors, to estimate parameters for the model and compute a linear combination of components using the estimated parameters.

33. A computer readable medium providing the computer program of claim 32.

34. An apparatus for identifying components from a system which exhibit a response pattern associated with test conditions applied to the system, and wherein a matrix of design factors to specify the type of response patterns for the set of tests and conditions is defined, the apparatus including a calculation device for identifying linear combinations of components from the input data which correlate with the response pattern.

35. An apparatus for identifying components from a system which exhibit a preselected response pattern to a set of test conditions applied to the biotechnology array, wherein a matrix of design factors to specify the response pattern(s) for the test conditions is defined, the apparatus including a means for formulating a model for the residuals on a regression of the input array data on the design factors, means for estimating parameters for the model and means for computing a linear combination of components using the estimated parameters.

36. A computer program which includes instructions arranged to control a computing device to implement the method of claim 1.
